(19) United States
(12) Patent Application Publication
(10) Pub. No.: US 2017/0274063 A1
(43) Pub. Date: Sep. 28, 2017

(54) THERMOSTABLE, CHROMATOGRAPHICALLY PURIFIED NANO-VLP VACCINE

(71) Applicants: John Howard CARRA, Sabillasville, MD (US); Sina BAVARI, Frederick, MD (US); David HONE, Poolesville, MD (US); U.S ARMY MEDICAL RESEARCH INSTITUTE OF INFECTIOUS DISEASES DEPARTMENT OF THE ARMY, Frederick, MD (US)

(72) Inventors: John Howard Carra, Sabillasville, MD (US); Sina Bavari, Frederick, MD (US); David Hone, Poolesville, MD (US)

(73) Assignee: THE GOVERNMENT OF THE UNITED STATES AS REPRESENTED BY THE SECRETARY OF THE ARMY, Frederick, MD (US)

(21) Appl. No.: 15/508,719

(22) PCT Filed: Aug. 7, 2015
(86) PCT No.: PCT/US15/44203

§ 371 Date: Mar. 3, 2017

Related U.S. Application Data
(60) Provisional application No. 62/035,182, filed on Aug. 8, 2014.

(51) Int. Cl. A61K 39/12; G01N 30/86; G01N 30/02; A61K 9/51; A61K 39/295

(52) U.S. Cl. CPC A61K 39/12; A61K 9/5184; A61K 39/295; G01N 30/02; G01N 30/8617; A61K 2039/55561

(57) ABSTRACT

In this application is described a method for preparing nano-VLP composition, thereby permitting purification using chromatography and filtration. The nano-VLP composition has a more uniform size range of filovirus particles, roughly 230 nm diameter, allowing ease of manipulation of the composition, while retaining GP conformational integrity and the antigenic effectiveness of the vaccine. Additionally, the nano-VLP can be lyophilized without loss of nano-VLP structure, or GP immunogenicity. Lyophilized nano-VLP have greatly enhanced thermostability, allowing the creation of a filovirus nano-VLP vaccine without a cold chain requirement.

### INTRODUCTION

The filoviruses Ebola and Marburg are enveloped viruses causing lethal, hemorrhagic disease in humans and non-human primates (Feldman et al., 2003, Nat Rev Immunol 3:677-685). The virions exist in a mixture of morphologies, including “6”-shaped and filamentous particles. The filaments are 80-100 nm in width and can be several microns long (Beniac et al. 2012, PLoS One 7:e29608). The surface of the virions is covered in trimeric spikes of the glycoprotein (GP), while the VP40 protein forms a structural matrix underlying the viral membrane. Formation of virus-like particles (VLP) with shapes similar to authentic filoviruses can be induced by transfection into human or insect cell lines of the genes for GP and VP40 alone (U.S. Pat. No. 7,682,618; Warfield et al., 2003, Proc Natl Acad Sci USA 100:15889-15894; Warfield et al., 2007, J Infect Dis 196 Supp12:S421-429; Warfield et al., 2005, Expert Rev Vaccines 4:429-440; Swenson et al., 2005, Vaccine 23:3033-3042). The five other viral proteins are not essential for production of virus-like particles, although some efforts have also included the nucleocapsid protein NP.

VLP are useful as laboratory reagents for the exploration of filovirus biology, and they are promising candidates for vaccines to protect humans against natural or deliberate exposure to these viruses (Martins et al., 2013, Virol Sin 28:65-70). Non-human primates have been successfully immunized against Ebola with VLP plus adjuvant, with at least two doses needed for full protection, while one conferred partial protection (Warfield et al., 2007, J Infect Dis 196 Suppl 2:S430-437; Warfield et al., 2015, PLoS One 10:e0118881). Filovirus VLP are more effective than soluble GP proteins at stimulating the immune system (Wahl-Jensen et al., 2005, J Virol 79:2413-2419). The presentation of GP trimers in a repetitive array likely contributes to their potency by increasing interactions with receptors on B cells and antigen-presenting cells (Beniac et al., supra; Bachmann and Jennings 2010, Nat Rev Immunol 10:787-796). However, unlike VLP of some other types of viruses, which may consist only of smaller, spherical and non-enveloped proteinaceous particles that can be reassembled in vitro from isolated subunits, filovirus VLP can be several microns in length and are enveloped. These characteristics present difficult problems in purification, sterilization and analytical methods. Due to the size of the VLP, development of the filovirus VLP as vaccines for humans hence has been limited by the methods used in production, which rely upon roduction of the VLP by transient transfection, sucrose gradient ultracentrifugation for purification and gamma-irradiation for sterilization. These methods are inefficient and introduce high costs to supply the doses used in non-human primate experiments (typically 50-250 μg GP).

Designing a smaller VLP that can be purified and sterilized using methods that are less costly and more efficient may resolve these drawbacks. The optimal size and shape of nanoparticle vaccines is an important factor in their design and a subject of current interest (reviewed in Zhao et al., 2014, Vaccine 32:327-337; Ungaro et al., 2013, Expert Rev Vaccines 12:1173-1193; Silva et al., 2013, J Control Release 168:179-199). For example, Manolova et al. (2008, Eur J Immunol 38:1404-1413) studied the effect of particle size on antigen uptake by dendritic cells and found that polystyrene beads of ≦200 nm were able to drain rapidly to lymph nodes, where they were taken up by lymph node-resident dendritic cells and macrophages. Beads ≧500 nm could not directly enter the lymphatic system, and were taken up more slowly by a different population of dendritic cells at the site of injection. Champion and Mitragotri (2009, Pharm Res 26:244-249) found that worm-like polystyrene particles were taken up by macrophages very poorly when compared to spherical particles. Even though these results suggest that it may be advantageous to the immune response to reduce the length of filovirus VLP, the influence of the large size of the filaments and the presentation of the GP antigen as part of the large VLP on stimulation of the immune response is not known.

Therefore, there is a need for better methods for preparation, purification and sterilization of VLP vaccines that retain their antigenic integrity.

### SUMMARY OF THE INVENTION

The present invention satisfies the needs described above.

In this application is described a new version of the Ebola VLP, nano-VLP (nVLP), consisting of smaller particles that are more uniform. The smaller size of the nano-VLP allows use of chromatography for purification and filtration. Surprisingly, the nano-VLP retains GP conformational integrity and the antigenic effectiveness of the vaccine even after lyophilization.

These results were unexpected since it was believed that changes in preparation of the VLP would result in a nonimmunogenic composition, i.e. that intact filaments were essential to VLP immunogenicity, and that by subjecting the VLP to harsh treatments, i.e. sonication, filter chromatography, and lyophilization, any of these steps, alone or in combination, would denature GP thereby drastically reducing or eliminating the effectiveness of the vaccine.

However, the inventors were able to optimize sonication procedures such that the nano-VLP produced were roughly 230 nm in average size and retained their efficacy as a vaccine in a mouse model. Using a combination of mouse bioassays, electron microsocopy (EM), antibody-based probe of GP, and a nanopore sizing method the inventors were able to produce a filter purified nano-VLP with no significant difference in potency from the large filamentous VLP.

Therefore, it is one object of the invention to provide a composition comprising nano-VLP. The nano-VLP consist of GP-coated particles in a mixture of morphologies including circular, branched, “6”-shaped, and filamentous. Intact VLP filaments can be several microns long. The nano-VLP filament fragments are usually less than 1 micron, with the more spherical particles being about 230 nm diameter. The nano-VLP can be further lyophilized to produce a nano-VLP powder. The nano-VLP solution or powder can be used in a diagnostic assay or as a vaccine, with or without adjuvant. Even though Ebola nano-VLP is described herein, a Marburg nano-VLP composition is also encompassed in this invention.

It is another object of the invention to provide a vaccine for inducing a protective immune response to a filovirus, namely Ebola or Marburg, said vaccine comprising Ebola nano-VLP or Marburg nano-VLP, respectively, or a combination of Ebola and Marburg nano-VLP.

It is yet another object of the invention to provide an immunological composition for inducing an immune response in a subject against Ebola or Marburg virus infection comprising Ebola or Marburg nano-VLP.

It is further another object of the invention further to provide a method for preparation of filovirus nano-VLP comprising isolating VLP from cells transfected with at least filovirus GP and VP40, sonicating the isolated VLP to produce sonicated VLP, and subjecting the sonicated VLP to filter chromatography to produce nano-VLP. The nano-VLP can optionally be lyophilized to produce lyophilized or powdered nano-VLP.

Advantageously, the lyophilized nano-VLP composition of the invention is thermostable. Ebola VLP underwent denaturation of GP when heated in liquid suspension to 75° C. for 15 min, which resulted in a nearly complete loss of protective capability in a mouse model. However, lyophilized nano-VLP could be heated in the vial before resuspension to 75° C. for at least 1 h, with little apparent loss of GP conformation as determined by conformational ELISA. The resuspended nano-VLP with or without adjuvant was protective in a mouse Ebola challenge.

Therefore, it is another object of the invention to provide lyophilized nano-VLP powder for use as a vaccine or a diagnostic agent.

It is another object of the present invention to provide a method for encapsulating desired agents into filovirus nano-VLP, e.g., therapeutic or diagnostic agents.

It is yet another object of the invention to provide filovirus nano-VLP, preferably Ebola nano-VLP or Marburg nano-VLP, which contain desired therapeutic or diagnostic agents contained therein, e.g. anti-cancer agents or antiviral agents. The nano-VLP are useful as a delivery agent for transferring into a cell a desired antigen or nucleic acid which would be contained in the internal space provided by the virus-like particles. When the desired antigen forms part of the virus particle, a hybrid, multi-agent nano-VLP is formed.

It is still another object of the invention to provide a novel method for delivering a desired moiety, e.g. a nucleic acid to desired cells wherein the delivery vehicle for such moiety, comprises filovirus nano-VLP.

It is another object of the present invention to produce a hybrid, multi-immunogen nano-VLP wherein the desired antigen or immunogen forms part of the virus particle and is displayed on the surface of the nano-VLP, wherein the antigen is a non-naturally occurring antigen in the virion of the native virus. The hybrid multi-immunogen nano-VLP and compositions comprising said hybrid, multi-immunogen nano-VLP can be used as an immunological composition for inducing an immune response against the filovirus and the desired antigen, as a multimeric vaccine protective against both the filovirus and the agent from which the antigen is derived, as a delivery vehicle and in a diagnostic assay.

It is another object of the invention to produce a vaccine for inducing an immune response to not only a filovirus, namely Ebola or Marburg, but also to another agent or pathogen, said vaccine comprising a hybrid, multi-immunogen nano-VLP wherein the nano-VLP is formed with a desired antigen or peptide from said agent or pathogen.

It is another object of the invention to provide a diagnostic assay for the detection of an agent in a sample from a subject suspected of having a disease. The disease can be from an infectious agent, a tumor agent, or an allergen.

The method comprises detecting the presence or absence of a complex formed between a hybrid nano-VLP having an immunogen or antigen found in said agent and anti-immunogen or anti-antigen antibodies in the sample.

The present invention further provides a rapid in vitro test for testing antigenic integrity of the GP as a correlate to in vivo potency. The usual test for the antigenic integrity of the GP is the mouse potency assay, which takes more than one month and involves all the costs associated with animal assays. The conformational ELISA described herein is a quick surrogate for the mouse assay. The method comprises detecting the presence or absence of a complex formed between anti-GP antibodies that bind linear epitopes and anti-GP antibodies that bind conformational epitopes, and comparing the amount of complexes formed using the different antibodies, such that the presence of an equal amount of complexes from both antibodies indicates antigenic integrity, and a reduced amount of complexes formed with the antibodies which bind the conformational epitope indicates conformational instability of GP or reduced immunogenicity of the GP antigen.

It is another object of the invention to provide a method and test kits for detection of Ebola or Marburg infections by detecting the presence of Ebola or Marburg antibodies in a sample from a subject suspected of having such an infection. The method comprises detecting the presence or absence of a complex formed between anti-Ebola antibodies, or anti-Marburg antibodies in the sample and Ebola nano-VLP or Marburg nano-VLP, respectively, such that presence or absence of the immunological complex(es) correlates with presence or absence of the respective infection. The nano-VLP can be directly or indirectly attached to a suitable reporter molecule, e.g., an enzyme, a radionuclide, or a fluorophore. The test kit includes a container holding one or more nano-VLP according to the present invention and instructions for using the nano-VLP for the purpose of detecting Ebola antibodies and/or Marburg antibodies in a sample.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

These and other features, aspects, and advantages of the present invention will become better understood with reference to the following description and appended claims, and accompanying drawings where:

FIGS. 1A and 1B. Electron micrographs of VLP before and after sonication. Bar is 0.5 μm. (A) Intact VLP. (B) Sonicated VLP.

FIG. 2A-D. Nanopore measurements. (A) Intact VLP (squares) sonicated VLP (triangles) and 217 nm polystyrene beads (circles.) (B) VLP after sonication and passage through a 0.45 μm (circles) or 0.8/0.2 μm (squares) filter. (C) The calculated particle size distribution of filtered VLP samples after calibration using the polystyrene beads. Black bars—0.45 μm, white bars—0.8/0.2 μm. (D) Pressure dependence of the particle flow rate. Calibration beads (triangles, dashed line), sonicated and filtered (0.45 mm) VLP (circles, dotted line). R² was 0.99 for both linear regressions.

FIG. 3A-C. Accelerated degradation of VLP. (A) Undisrupted VLP incubated at elevated temperatures for the times indicated and probed by antibodies 6D8 (black bars), 6D3 (white bars), or 13C6 (gray bars). The absorbance at 408 nm is shown on the y-axis. (B) Sonicated VLP, same as part A. (C) Undisrupted VLP were subjected to repeated rounds of freeze/thawing in the presence or absence of 5% sucrose.

FIG. 4A-C. Impact of heating or sonication on VLP immunogenicity. (A) Schematic depicting vaccination schedule. Mice were vaccinated on days 0 and 21. Blood was collected on day 35, followed by challenge on day 49, with the end of study on day 63. (B) C57BL/6 mice were vaccinated two times, with three weeks between vaccinations. VLP were untreated, heated at 37° C. for 96 h, heated at 75° C. for 15 min, or sonicated. Mice were challenged four weeks after the final vaccination and the percent survival is shown. p-values determined using Fisher's exact test to compare survival of each treatment group at the given dose level to control VLP (“untreated”). (C) Anti-glycoprotein antibody titers of vaccinated mice measured from serum collected two weeks after the final vaccination. VLP dose level (10 or 2.5 μg) and treatment (untreated, heating conditions, and sonication status) are indicated on the x-axis. P values determined using one-tailed Student's t-test where * indicates p<0.05, ** indicates p<0.005, *** indicates p<0.0005. Legend for both B and C: Black circles, saline, dose level 0; solid red triangle, untreated, dose level 10 ug; empty red triangle, untreated, dose level 2.5 ug; solid blue circle, 37° C. heated, dose level 10 ug; empty blue circle, 37° C. heated, dose level 2.5 ug; inverted empty orange triangle, 75° C. heated, dose level 10 ug; solid orange inverted triangle, 75° C. heated, dose level 2.5 ug; solid purple circles, sonicated, dose level 10 ug; empty purple circles, sonicated, dose level 2.5 ug.

FIGS. 5A and 5B. Impacts of sonication and filtration on VLP immunogenicity. (A) C57BL/6 mice were vaccinated two times, with three weeks between vaccinations, as in FIG. 4A. VLP were untreated, sonicated, or sonicated and passed through either a 0.45 μm or 0.8/0.2 μm filter (indicated on x-axis). The percent survival is shown. p-values determined using Fisher's exact test to compare survival of each treatment group at the given dose level to control VLP (“untreated”). (B) Anti-glycoprotein antibody titers of vaccinated mice measured from serum collected two weeks after the final vaccination. VLP dose level (10 or 20 μg) is indicated on the x-axis. P values determined using one-tailed Student's t-test where * indicates p<0.05, ** indicates p<0.005, *** indicates p<0.0005. Legend for both A and B: Solid black circle, saline, dose level 0; empty red triangle, untreated, dose level 20 ug; solid red triangle, untreated, dose level 10 ug; empty purple circle, sonicated, dose level 20 ug; solid purple circle, sonicated 10 ug; inverted empty blue triangle, sonicated and filtered 0.45 um, dose level 20 ug; inverted solid blue triangle, sonicated and filtered 0.45 um, dose level 10 ug; empty grey diamond, sonicated and filtered 0.8/0.2 um, dose level 20 ug; solid gray diamond, sonicated and filtered 0.8/0.2 um, dose level 10 ug.

FIG. 6A-D. Electron micrographs. (A) nano-VLP. Bar=2 μm. (B) Lyophilized nano-VLP, after resuspension. Bar=0.5 μm. (C) Lyophilized nano-VLP. Bar=0.1 μm. D. Lyophilized nano-VLP after heating to 75° C. for 1 h. Bar=0.1 μm.

FIGS. 7A and 7B. (A) Nanopore event duration (ordinate) and blockade magnitude (abscissa) of non-lyophilized (circles) and lyophilized (triangles) nano-VLP. (B) ELISA to probe the conformational integrity of GP in nano-VLP that were not lyophilized, lyophilized, and lyophilized and heated to 75° C. for 1 h. Black bars—linear epitope antibody 6D8, white bars—conformational antibody 6D3, gray bars—conformational antibody 13C6.

FIG. 8A-D. (A) C57BL/6 mice were vaccinated two times, with three weeks between vaccinations, as in FIG. 4A. Dose level of VLP or nano-VLP (nVLP) was 5 μg, based on GP content, and dose level of the adjuvant poly-ICLC was 10 μg. Time to death in days, expressed as percentage survival in each group. All animals except those in the adjuvant-only poly-ICLC control group survived. Black line, saline+adjuvant; gray line, sucrose-purified VLP+adjuvant; blue line, nVLP; purple line, lyophilized VLP+adjuvant; orange line, lyophilized nVLP, heated+adjuvant. (B) Anti-glycoprotein titers of mice from A inoculated with adjuvant alone (black diamonds), or adjuvant plus sucrose-gradient control VLP (gray diamonds), adjuvant+frozen nVLP (blue diamonds), adjuvant+lyophilized nVLP (purple diamonds), and adjuvant+lyophilized nVLP heated to 75° C. for 1 h (orange diamonds). (C) Time to death in days, expressed as percentage survival in each group of mice vaccinated with 5 or 20 μg nVLP doses, without adjuvant. The experiment compares results for nVLP stored frozen, lyophilized nVLP, lyophilized and heated nVLP, and a control of sucrose-gradient purified VLP. p-values determined using Fisher's exact test to compare survival of each treatment group vs. saline. Black solid diamond, saline; blue line, nVLP, dose level 5 ug; blue triangle, nVLP, dose level 20 ug; purple line, lyophilized nVLP, dose level 5 ug; purple diamond, lyophilized nVLP, dose level 20 ug; orange line, lyophilized nVLP, heated, dose level 5 ug; solid orange circles, lyophilized nVLP, heated, dose level 20 ug. (D) Anti-glycoprotein titers of mice from C. P values determined using one-tailed Student's t-test where * indicates p<0.05, ** indicates p<0.005, *** indicates p<0.0005. Solid black diamonds, saline; solid blue diamonds, nVLP 5 ug; blue-filled black diamonds, nVLP, dose level 20 ug; solid purple diamonds, lyophilized nVLP, dose level 5 ug; purple-filled black diamonds, lyophilized nVLP, dose level 20 ug; solid orange diamonds, lyophilized nVLP heated, dose level 5 ug; orange filled black diamonds, lyophilized nVLP heated, dose level 20 ug.

### DETAILED DESCRIPTION

In the description that follows, a number of terms used in recombinant DNA, virology and immunology are extensively utilized. In order to provide a clearer and consistent understanding of the specification and claims, including the scope to be given such terms, the following definitions are provided.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It also is specifically understood that any numerical value recited herein includes all values from the lower value to the upper value, i.e., all possible combinations of numerical values between the lowest value and the highest value enumerated are to be considered to be expressly stated in this application. For example, if a range is stated as 1% to 50%, it is intended that values such as 2% to 40%, 10% to 30%, or 1% to 3%, etc., are expressly enumerated in this specification.

“Contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or polysaccharide or an epitope on a particular polypeptide or polysaccharide is one that binds to that particular polypeptide or polysaccharide or epitope on a particular polypeptide or polysaccharide without substantially binding to any other polypeptide or polypeptide epitope.

Polyclonal antibodies are immunoglobulin molecules that react against a specific antigen, each antibody identifying a different epitope on the antigen. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

Monoclonal antibodies are immunoglobulin molecules that recognize a specific epitope on a specific antigen.

Monoclonal antibodies may be prepared using hybridoma methods, such as those described by Kohler and Milstein, Nature, 256:495 (1975). Other methods of preparing monoclonal antibodies are well known in the art. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the polypeptide or polysaccharide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the desired antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, 1980, Anal. Biochem., 107:220.

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., 1995, Protein Eng. 8(10): 1057-1062); single-chain antibody molecules; and multispecific antibodies formed from antibody fragments.

“Nucleic acid,” “oligonucleotide,” and “polynucleotide” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. The term nucleic acid is used interchangeably with gene, cDNA, and mRNA encoded by a gene.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

The word “label” when used herein refers to a detectable compound or composition which is conjugated directly or indirectly to the antibody so as to generate a “labeled” antibody or to a nano-VLP to generate a “labeled” nano-VLP. The label may be detectable by itself (e.g. radioisotope labels or fluorescent labels) or, in the case of an enzymatic label, may catalyze chemical alteration of a substrate compound or composition which is detectable.

The word “subject” includes human, animal, avian, e.g., horse, donkey, pig, mouse, hamster, monkey, chicken, sheep, cattle, goat, buffalo, and any other subject suspected of being infected with Ebola or Marburg virus.

The language “biological sample” is intended to include biological material, e.g. cells, blood, tissues, biological fluid, or a solution for administering to a subject, such as a vaccine, or immunoglobulin. By “environmental sample” is meant a sample such as soil and water. Food samples include canned goods, meats, milk, and other suspected contaminated food. Forensic sample includes any sample from a suspected terrorist attack, including paper, powder, envelope, container, hair, fibers, and others.

“Dry” in the context of freeze drying or lyophilization, refers to residual moisture content less than about 10%. Dried compositions are commonly dried to residual moistures of 5% or less, or between about 3% and 0.1%.

Lyophilization (or freeze-drying) is a dehydration technique in which the sample solution (e.g., a nano-VLP composition) is frozen and the solvent (e.g., water or buffer) is removed by sublimation by applying high vacuum. The technique of lyophilization is well known to one of skill in the art (Rey and May, 1999).

“Excipients” or “protectants” (including cryoprotectants and lyoprotectants) generally refer to compounds or materials that are added to ensure or increase the stability of the therapeutic agent during the dehydration processes, e.g. foam drying, spray drying, freeze drying, etc., and afterwards, for long term stability.

A “stable” formulation or composition is one in which the biologically active material therein essentially retains its physical stability and/or chemical stability and/or biological activity upon storage. Stability can be measured at a selected temperature for a selected time period. Trend analysis can be used to estimate an expected shelf life before a material has actually been in storage for that time period.

“Pharmaceutically acceptable” refers to those active agents, salts, and excipients which are, within the scope of sound medical judgment, suitable for use in contact with the tissues or humans and lower animals without undue toxicity, irritation, allergic response and the like, commensurate with a reasonable benefit/risk ratio, and effective for their intended use.

Filoviruses. The filoviruses (e.g. Ebola virus (EBOV) and Marburg virus (MBGV)) cause acute hemorrhagic fever characterized by high mortality. Humans can contract filoviruses by infection in endemic regions, by contact with imported primates, and by performing scientific research with the virus. However, there currently are no available vaccines or effective therapeutic treatments for filovirus infection. The virions of filoviruses contain seven proteins which include a surface glycoprotein (GP), a nucleoprotein (NP), an RNA-dependent RNA polymerase (L), and four virion structural proteins (VP24, VP30, VP35, and VP40).

Virus-like particles (VLP). This refers to a structure which resembles the outer envelope of the native virus antigenically and morphologically. The virus-like particles are formed in vitro upon expression, in a cell, of viral surface glycoprotein (GP) and a virion structural protein, VP40. It may be possible to produce VLPs by expressing only portions of GP and VP40. Methods of making and using VLP are described in U.S. Pat. No. 7,682,618 issued on Mar. 23, 2010. All references cited herein are hereby incorporated in their entirety by reference thereto.

Formation of Ebola and Marburg VLP is dependent on lipid rafts, tightly regulated specialized domains in the cell membrane. Lipid raft components are targets for therapeutic interventions (Bavari et al., 2002, J Exp Med 195:593-602; Warfield and Aman, 2011, J Infect Dis 204 Supp13:S1053-9). Therefore, VLPs are useful as vaccines against filovirus infections, and as vehicles for the delivery to cells of a variety of antigens artificially targeted to the rafts.

The intact VLP vary in size and have different shapes (Bavari et al., 2001, J Exper Med 195:593-602), and are several microns in length and present problems in purification, sterilization and analytical methods.

The present invention relates to nano-VLP, or nVLP, and a method of producing nano-VLP from intact filovirus VLP. The method includes expressing viral glycoprotein GP and the virion structural protein, VP40 in cells to produce VLP.

nano-VLP consist of GP-coated virus-like particles which are smaller than intact VLP. In contrast to intact filovirus VLP, which contain filaments that are several microns in length and are therefore difficult to manipulate, nano-VLP consist of shorter filaments of about 500 nm in length, and spherical particles of about 230 nm diameter. The reduced-size VLP are easily purified and can be filtered to remove larger aggregates of cell debris and bacterial contaminants, thereby reducing bioburden. Therefore, the filtered nano-VLP are a much purer preparation than intact VLP. nano-VLP retain temperature stability, the structure of the GP antigen, and the ability to stimulate a protective immune response in mice.

In some embodiments, the present invention provides nano-VLP in a mixture of spherical nano-VLP in spherical particles and filamentous nano-VLP. Therefore, the present invention provides spherical nano-VLP having from about from about 20 nm to about 500 nm in diameter, or from about 50 to about 450 nm in diameter, or from about 100 to about 400 nm in diameter, or from about 150 to about 350 nm in diameter, or from about 200 to about 250 nm in diameter, or from about 200 to about 300 nm in diameter, or from about 100 to about 200 in diameter, or from about 200 to about 400 nm in diameter, or from about 100 nm to about 500 nm in diameter, or from about 150 nm to about 450 nm in diameter, or from about 200 nm to about 250 nm in diameter. The present invention provides filamentous nano-VLP having from about 20 nm to about 1500 nm in length, or from about 50 to about 1200 nm in length, or from about 100 to about 1000 nm in length, or from about 200 to about 800 nm in length, or from about 300 to about 700 nm in length, or from about 400 to about 600 nm in length, or from about 400 to about 500 in length, or from about 200 to about 500 nm in length, or from about 100 nm to about 500 nm in length, or from about 150 nm to about 450 nm in length, or from about 300 nm to about 1000 nm in length.

In another embodiment, the purified nano-VLP are produced by

isolating intact VLP from cells transfected with one or more expression vector expressing filovirus GP and VP40;

deaggregating the isolated VLP by ultrasound or sonication to produce sonicated VLP or nano-VLP; and

purifying the nano-VLP to produce purified nano-VLP.

Further purification and isolation steps resulting in only spherical nVLP and/or only filamentous nVLP are envisioned.

Production of VLP has been described elsewhere. Briefly, VLP are produced by expressing viral glycoprotein GP and the virion structural protein, VP40 in cells by transfection of DNA fragments which encode these proteins into the desired cells. DNA fragments which encode any of the Ebola Zaire 1976 or 1995 (Mayinga isolate) GP and VP40 proteins are known. Accession# AY142960 contains the whole genome of Ebola Zaire, with individual genes including GP and VP40 specified in this entry, VP40 gene nucleotides 4479-5459, GP gene 6039-8068. The entire Marburg genome has been deposited in accession # NC_001608 for the entire genome, with individual genes specified in the entry, VP40 gene 4567-5478, GP gene 5940-7985, NP gene 103-2190. The protein ID for Ebola VP40 is AAN37506.1, for Ebola GP is AAN37507.1, for Marburg VP40 is CAA78116.1, and for Marburg GP is CAA78117.1. The DNA fragments can be inserted into a mammalian expression vector and transfected into cells.

The filovirus gene products can be expressed in eukaryotic host cells such as yeast cells and mammalian cells. Saccharomyces cerevisiae, Saccharomyces carlsbergensis, and Pichia pastoris are the most commonly used yeast hosts. Control sequences for yeast vectors are known in the art. Mammalian cell lines available as hosts for expression of cloned genes are known in the art and include many immortalized cell lines available from the American Type Culture Collection (ATCC), such as HEPG-2, CHO cells, Vero cells, baby hamster kidney (BHK) cells and COS cells, to name a few. Suitable promoters are also known in the art and include viral promoters such as that from SV40, Rous sarcoma virus (RSV), adenovirus (ADV), bovine papilloma virus (BPV), and cytomegalovirus (CMV). Mammalian cells may also require terminator sequences, poly A addition sequences, enhancer sequences which increase expression, or sequences which cause amplification of the gene. These sequences are known in the art.

Cells may be transfected with one or more expression vector expressing filovirus GP and VP40 using any method known in the art, for example, calcium phosphate transfection as described in the examples. Any other method of introducing the DNA such that the encoded proteins are properly expressed can be used, such as viral infection, and electroporation, to name a few.

The transformed or transfected host cells can be used as a source of the intact VLP for producing the nano-VLP described below.

The nano-VLP of the present invention can be prepared by any suitable method known to one of skill in the art. For example, in a more specific embodiment, the nano-VLP were prepared as follows. Host cells transformed with one or more expression vector expressing filovirus GP and VP40 were allowed to grow for three days or until the cells die, after which intact VLP were isolated by removing cells by centrifugation. The VLP pellet was resuspended in 10 mM sodium phosphate and 50 mM NaCl, pH 7.4 and kept on ice. Other buffers similar to PBS can be used.

The solution was sonicated to increase the fluidity of the sample, deaggregate the intact VLP, and decrease the length of filamentous VLP to allow for more efficient filtration. Other methods may be used, such as microfluidization, but care must be taken not to damage the VLP. By sonication or ultrasound processing is meant the application of sound energy to agitate particles in solution. This is usually applied using a sonicator. The result is deagglomeration of molecules and even dispersement of molecules. Once parameters have been defined for laboratory use, industrial scale-up for continuous production is possible. For guidance, please see Peshkovsky et al., 2013, Chemical Engineering and Processing: Process Intensification, 69, p. 77-62, and A. S. Peshkovsky, S. L. Peshkovsky “Industrial-scale processing of liquids by high-intensity acoustic cavitation—the underlying theory and ultrasonic equipment design principles”, In: Nowak F. M, ed., Sonochemistry: Theory, Reactions and Syntheses, and Applications, Hauppauge, N.Y.: Nova Science Publishers; 2010.

Sonication of VLP was done using a Branson Sonifier 250 as described in the Examples below. Varying numbers of pulses were tested and monitored using the conformation ELISA and nanopore sizing methods until the proper results were achieved. Briefly, 10-12 mL of VLP were sonicated for 4 sets of 3 pulses of 1 sec duration, at 50% duty cycle with the output control at 5.5. Samples were chilled on ice after each set of pulses. Sonication was applied until the resulting nano-VLP were of characteristic size as described above, while retaining the GP integrity as determined by the conformational ELISA.

After sonication, samples were purified by filtration. Filtration is used to purify or concentrate samples, and for size-exclusion chromatography. A solution is passed though a filter or other material that prevents passage of certain molecules, particles, or substances. Considerations for choosing a filter include loading capacity, particle retention efficiency or particle size (usually in um), fluid flow rate through the filter and pore size. Many filter types exist, such as filter papers, glass microfiber filters, and membrane filters, to name a few. Selecting the right filter is by trial and error and specifications and guidance on filter choice is provided by the filter manufacturer.

After sonication the samples were prepared for filtration by dilution with an equal volume of buffer to aid in passing through the filter and reduce dead-volume loss. The solution was filtered to remove yellow colloidal material or cell debris. Filtration included once through a 2.5 cm GF/D filter (Whatman glass microfiber), and then twice through GF/F filters, or until the solution can pass through the next filtration step.

20 mL of the filtered sample was then run over a Sartorius Sartobind S-75 membrane chromatography disc, pre-equilibrated with buffer on a GE Healthcare FPLC system. This filtration retains contaminants while allowing the nano-VLP to pass through. The VLP flowed through the S-75 disc at a flow rate of 1 mL/min at a backpressure of 0.16 MPa, or until all the material flows through the disc and the contaminants are removed.

The flow through of the S-75 disc was again sonicated for 3 sets of 3 pulses in order to confirm that the particles are small enough to pass through the next filtration.

The sample was then centrifuged and the pellet suspended in buffer solution with 5% trehalose. Other lyoprotectants can be used as long as the lyophilized nVLP powder or the resuspended lyophilized nVLP retains GP antigenicity. The final filtration step to remove aggregates was done with 2.5 cm Pall 0.45 uM Supor filter. Other filters can be used, e.g. a 0.8/0.2 micron Supor filter, however, as long as there is no additional loss of material and the results from the GP conformational testing is positive.

For lyophilization, 262 uL volume samples in buffer with 5% trehalose containing 60 ug of GP (as part of the nano-VLP) were frozen on dry ice and then placed on a shelf of a VirTis AdVantage ES freezedryer at −20° C. for drying in one stage. After 24 h exposure to vacuum (80 MT), the samples were reduced to a white powder. Other lyophiliztion methods known in the art can be used as long as the resuspended nano-VLP powder results in nano-VLP of the present invention.

While these results are novel and unexpected, based on the teachings of this application, one skilled in the art may achieve greater nano-VLP yields by varying conditions of VLP isolation, deaggregation, and purification.

The nano-VLPs are comprised of GP and VP40. Other proteins can be added when designing the VLP such as NP, VP24, VP30, and VP35 without affecting the structure of the resulting nVLP. In the case of a hybrid nano-VLP, the expressed VLP will additionally contain a desired antigen or part of an antigen.

nano-VLPs can also be produced from intact VLP using more than one GP or VP40 from different filoviruses or filovirus strains. When portions of GP from different filoviruses are combined or fused to form one GP protein, the VLP expressing this fusion protein is chimeric. A chimeric nano-VLP produced from a chimeric VLP can comprise, for example, GP1 from one filovirus fused to GP2 from a different filovirus, or portions of GP1 and GP2 from more than two filoviruses such that a complete GP protein is expressed. The source of GP1 and GP2 can be a different filovirus, i.e. Ebola or Marburg, or it can be different strains or species of the same filovirus, i.e. Ebola Sudan and Ebola Zaire.

All filoviruses have GP proteins that have similar structure, but with allelic variation. By allelic variation is meant a natural or synthetic change in one or more amino acids which occurs between different serotypes or strains of Ebola or Marburg virus and does not affect the antigenic properties of the protein. There are different strains of Ebola (Zaire 1976, Zaire 1995, Reston, Sudan, and Ivory Coast with 1-6 species under each strain). Marburg has species Musoke, Ravn, Ozolin, Popp, Ratayczak, Voege. The GP and VP genes of these different viruses have not been sequenced. It would be expected that these proteins would have homology among different strains. It is reasonable to expect that similar nano-VLP from other filoviruses can be prepared by using the concept of the present invention described for Ebola, i.e. expression of GP and VP40 genes from other filovirus strains would result in VLPs specific for those strains from which nano-VLP can be produced.

In another embodiment, the present invention relates to hybrid multi-immunogen nano-VLP wherein the VLP is formed as a hybrid molecule or fusion molecule. The term “hybrid molecule” and “fusion molecule” refers to a molecule in which two or more subunit molecules are linked, either covalently or non-covalently. The subunit molecules can be the same chemical type of molecule, or can be different chemical types of molecules. Thus, as used herein, the term refers to any molecule containing a filovirus protein or peptide and at least one immunogen not naturally associated with the source filovirus. By “source filovirus” is meant the filovirus from which the VLP-associated protein(s), or VLP forming protein, is derived. Other filovirus immunogens not from the source filovirus can be used to form a multi-immunogen VLP and then nano-VLP.

The subunit molecules forming the fusion molecules include, but are not limited to, fusion polypeptides (for example, a fusion between a filovirus polypeptide and an immunogen polypeptide) and fusion nucleic acids (for example, a nucleic acid encoding the fusion polypeptide). When used in reference to proteins, the terms “fusion protein” or “fusion polypeptide” refer to polypeptides in which filovirus amino acid sequences (e.g., GP sequences) and one or more immunogen polypeptides are expressed in a single protein.

By “multi-immunogen” nano-VLP is meant a nano-VLP with more than one immunogen is found on the surface of the nano-VLP. The first immunogen can be a filovirus immunogen. However, if an immune reaction to the source filovirus is not desired, multi-immunogen can refer to multiple non-filovirus immunogens. This is possible since the only requirement for VLP formation is VP40, and since the receptor binding domain of GP must be present in order to induce protective immune response to the filovirus.

As a means for forming multi-immunogen nano-VLPs containing immunogens not naturally associated with a source filovirus VLP, a linkage may be formed between a VLP-associated polypeptide and a desired immunogen. Multiple antigens, or peptides from multiple antigens, can be expressed by fusing or linking the antigen(s) such that they are expressed in the VLP. The antigens can be arranged in tandem, or each fused to different VLP-associated proteins or polypeptides. The filovirus polypeptide(s) may be flanked on one or both sides by the desired immunogen or immunogens.

All of the naturally occurring filovirus proteins can be used keeping in mind that VP40 is important for proper formation of the VLP structure. The VLP-associated polypeptide may be linked to a single immunogen or to multiple immunogens to increase immunogenicity of the VLP and nano-VLP, to confer immunogenicity to various pathogens, or to confer immunogenicity to various strains of a particular pathogen.

The linkage between the immunogen and a VLP-associated polypeptide can be any type of linkage sufficient to result in the immunogen being incorporated into the VLP. The bond can be a covalent bond, an ionic interaction, a hydrogen bond, an ionic bond, a van der Waals force, a metal-ligand interaction, or an antibody-antigen interaction. In certain embodiments, the linkage is a covalent bond, such as a peptide bond, carbon-oxygen bond, a carbon-sulfur bond, a carbon-nitrogen bond, a carbon-carbon bond, or a disulfide bond.

The immunogen may be produced recombinantly with an existing linkage to the VLP-associated polypeptide or it may be produced as an isolated substance and then linked at a later time to the VLP-associated polypeptide.

The immunogens as used herein can be any substance capable of eliciting an immune response. Immunogens include, but are not limited to, proteins, polypeptides (including active proteins and individual polypeptide epitopes within proteins), glycopolypeptides, lipopolypeptides, peptides, polysaccharides, polysaccharide conjugates, peptide and non-peptide mimics of polysaccharides and other molecules, small molecules, lipids, glycolipids, and carbohydrates.

The immunogen can be from any antigen implicated in a disease or disorder, e.g., microbial antigens (e.g., viral antigens, bacterial antigens, fungal antigens, protozoan antigens, helminth antigens, yeast antigens, etc.), tumor antigens, allergens and the like.

The immunogens described herein may be synthesized chemically or enzymatically, produced recombinantly, isolated from a natural source, or a combination of the foregoing. The immunogen may be purified, partially purified, or a crude extract.

Polypeptide immunogens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. In many embodiments, the immunogen is a purified antigen, e.g., from about 50% to about 75% pure, from about 75% to about 85% pure, from about 85% to about 90% pure, from about 90% to about 95% pure, from about 95% to about 98% pure, from about 98% to about 99% pure, or greater than 99% pure.

One may employ solid phase peptide synthesis techniques, where such techniques are known to those of skill in the art. See Jones, The Chemical Synthesis of Peptides (Clarendon Press, Oxford) (1994). Generally, in such methods a peptide is produced through the sequential additional of activated monomeric units to a solid phase bound growing peptide chain.

Well-established recombinant DNA techniques can be employed for production of polypeptides either in the same vector as the VLP-associated polypeptide, where, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

Suitable viral immunogens include those associated with (e.g., synthesized by) viruses of one or more of the following groups: Retroviridae (e.g. human immunodeficiency viruses, such as HIV-1 (also referred to as HTLV-III, LAV or HTLV-III/LAV, or HIV-III); and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis, including Norwalk and related viruses); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); and astroviruses.

Suitable bacterial immunogens include immunogens associated with (e.g., synthesized by and endogenous to) any of a variety of pathogenic bacteria, including, e.g., pathogenic gram positive bacteria such as pathogenic Pasteurella species, Staphylococci species, and Streptococcus species; and gram-negative pathogens such as those of the genera Neisseria, Escherichia, Bordetella, Campylobacter, Legionella, Pseudomonas, Shigella, Vibrio, Yersinia, Salmonella, Haemophilus, Brucella, Francisella and Bacterioides. See, e.g., Schaechter, M, H. Medoff, D. Schlesinger, Mechanisms of Microbial Disease. Williams and Wilkins, Baltimore (1989).

Suitable immunogens associated with (e.g., synthesized by and endogenous to) infectious pathogenic fungi include antigens associated with infectious fungi including but not limited to: Cryptococcus neoformans, Histoplasma capsulatum, Coccidioides immitis, Blastomyces dermatitidis, and Candida albicans, Candida glabrata, Aspergillus fumigata, Aspergillus flavus, and Sporothrix schenckii.

Suitable immunogens associated with (e.g., synthesized by and endogenous to) pathogenic protozoa, helminths, and other eukaryotic microbial pathogens include antigens associated with protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Suitable immunogens include antigens associated with (e.g., synthesized by and endogenous to) pathogenic microorganisms such as: Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Chlamydia trachomatis, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israeli. Non-limiting examples of pathogenic E. coli strains are: ATCC No. 31618, 23505, 43886, 43892, 35401, 43896, 33985, 31619 and 31617.

Any of a variety of polypeptides or other immunogens associated with intracellular pathogens may be included in the VLP and nano-VLP. Polypeptides and peptide epitopes associated with intracellular pathogens are any polypeptide associated with (e.g., encoded by) an intracellular pathogen, fragments of which are displayed together with MHC Class I molecule on the surface of the infected cell such that they are recognized by, e.g., bound by a T-cell antigen receptor on the surface of, a CD8+ lymphocyte. Polypeptides and peptide epitopes associated with intracellular pathogens are known in the art and include, but are not limited to, antigens associated with human immunodeficiency virus, e.g., HIV gp120, or an antigenic fragment thereof; cytomegalovirus antigens; Mycobacterium antigens (e.g., Mycobacterium avium, Mycobacterium tuberculosis, and the like); Pneumocystic carinii (PCP) antigens; malarial antigens, including, but not limited to, antigens associated with Plasmodium falciparum or any other malarial species, such as 41-3, AMA-1, CSP, PFEMP-1, GBP-130, MSP-1, PFS-16, SERP, etc.; fungal antigens; yeast antigens (e.g., an antigen of a Candida spp.); toxoplasma antigens, including, but not limited to, antigens associated with Toxoplasma gondii, Toxoplasma encephalitis, or any other Toxoplasma species; Epstein-Barr virus (EBV) antigens; Plasmodium antigens (e.g., gp190/MSP1, and the like); etc.

Any of a variety of known tumor-specific immunogens or tumor-associated antigens (TAA) can be included as an immunogen in the VLPs. The entire TAA may be, but need not be, used. Instead, a portion of a TAA, e.g., an epitope, may be used. Tumor-associated antigens (or epitope-containing fragments thereof) which may be used in VLPs include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV18, MAN, alpha-feto protein (AFP), OFP, CA-125, CA-50, CA-19-9, renal tumor-associated antigen G250, EGP-40 (also known as EpCAM), 5100 (malignant melanoma-associated antigen), p53, and p21ras. A synthetic analog of any TAA (or epitope thereof), including any of the foregoing, may be used. Furthermore, combinations of one or more TAAs (or epitopes thereof) may be included in the composition.

In one aspect, the immunogen that is part of the nano-VLP vaccine may be any of a variety of allergens. Allergen based vaccines may be used to induce tolerance in a subject to the allergen. Any of a variety of allergens can be included in VLP from which nano-VLP is produced. Allergens include but are not limited to environmental aeroallergens; plant pollens such as ragweed/hayfever; weed pollen allergens; grass pollen allergens; Johnson grass; tree pollen allergens; ryegrass; arachnid allergens, such as house dust mite allergens (e.g., Der p I, Der f I, etc.); storage mite allergens; Japanese cedar pollen/hay fever; mold spore allergens; animal allergens (e.g., dog, guinea pig, hamster, gerbil, rat, mouse, etc., allergens); food allergens (e.g., allergens of crustaceans; nuts, such as peanuts; citrus fruits); insect allergens; venoms: (Hymenoptera, yellow jacket, honey bee, wasp, hornet, fire ant); other environmental insect allergens from cockroaches, fleas, mosquitoes, etc.; bacterial allergens such as streptococcal antigens; parasite allergens such as Ascaris antigen; viral antigens; fungal spores; drug allergens; antibiotics; penicillins and related compounds; other antibiotics; whole proteins such as hormones (insulin), enzymes (streptokinase); all drugs and their metabolites capable of acting as incomplete antigens or haptens; industrial chemicals and metabolites capable of acting as haptens and functioning as allergens (e.g., the acid anhydrides (such as trimellitic anhydride) and the isocyanates (such as toluene diisocyanate)); occupational allergens such as flour (e.g., allergens causing Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates.

Examples of specific natural, animal and plant allergens include but are not limited to proteins specific to the following genera: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoas); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherun elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In another embodiment, the present invention relates to a single-component vaccine protective against filovirus. nano-VLP should be recognized by the body as immunogens but will be unable to replicate in the host due to the lack of appropriate viral genes, thus, they are promising as vaccine candidates. In a specific embodiment the filoviruses are MBGV and EBOV. A specific vaccine of the present invention comprises one or more nano-VLP derived from cells expressing EBOV GP, VP40, and potentially NP, VP24, VP30, and/or VP35 for use as an Ebola vaccine, or nano-VLP derived from cells expressing MBGV GP, VP40, and potentially NP, VP24, VP30 and/or VP35 for use as a Marburg vaccine. Hybrid nano-VLP produced by mixing GP and VP40 from two or more filoviruses are another embodiment of the present invention. For example, a hybrid nano-VLP can be produced using EBOV GP and Marburg VP40, or Marburg GP and EBOV VP40. Even though the specific strains of EBOV were used in the examples below, it is expected that protection would be afforded using nano-VLP from other EBOV strains and isolates, and/or other MBGV strains and isolates.

Hybrid, multi-immunogen VLPs comprising one or more non-naturally occurring filovirus immunogen can be used as a multi-agent vaccine in order to provide protection from a broad spectrum of agents simultaneously.

The present invention also relates to a method for providing immunity against MBGV and EBOV virus said method comprising administering one or more nano-VLP to a subject such that a protective immune reaction is generated. When protection against more than one filovirus is desired, a panfilovirus vaccine can be prepared. A panfilovirus vaccine can be prepared by mixing nano-VLP from different filoviruses, i.e. mixing Ebola nano-VLP and Marburg nano-VLP in a solution. Alternatively, a panfilovirus vaccine is comprised of one or more hybrid nano-VLP comprised of one or more GP or VP40, each from a different filovirus for which protection is desired.

In another embodiment, the present invention provides a method of inducing an immune response to a plurality of immunogens, e.g. two or more (e.g. 3, 4, 5, 6, 7 8 or more immunogens) derived from a plurality of pathogens from those described herein in a subject, comprising administering a multi-immunogen nano-VLP to the subject under conditions such that the subject produces an immune response.

This approach provides advantages over single agent vaccine in that the particle is multi-functional in terms of the plurality of immune, immune modulatory, and immune stimulatory peptides displayed on the surface of the VLP. This allows more efficient cellular uptake and processing resulting in reduced dose and improved immune protection.

Vaccine formulations of the present invention comprise an immunogenic amount of nano-VLP or a combination of nano-VLP as a multivalent vaccine, in combination with a pharmaceutically acceptable carrier. The nano-VLP vaccine may further comprise only spherical nano-VLP, or only filamentous nano-VLP, or a combination of spherical and filamentous chosen to produce the desired protective response. An “immunogenic amount” is an amount of the nano-VLP sufficient to evoke an immune response in the subject to which the vaccine is administered. An amount of from about 20 ug or 1.0 mg or more nano-VLP per dose with one to four doses one month apart is suitable, depending upon the age and species of the subject being treated. Exemplary pharmaceutically acceptable carriers include, but are not limited to, sterile pyrogen-free water and sterile pyrogen-free physiological saline solution.

nano-VLP of the present invention can be linked to other particles, such as gold nanoparticles and magnetic nanoparticles that are typically a few nanometers in diameter for imaging and manipulation purposes.

In another embodiment, the present invention relates to a method for producing nano-VLP which have encapsulated therein a desired moiety.

Loading of the diagnostic and therapeutic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006). In some embodiments, one or more therapeutic agents can be loaded into the nano-VLP. Loading of nano-VLP can be carried out, for example, in an active or passive manner. For example, a therapeutic agent can be included during the self-assembly process of the nano-VLP in a solution, such that the therapeutic agent is encapsulated within the nano-VLP. In certain embodiments, the therapeutic agent may also be embedded in the viral envelope or viral membrane. In alternative embodiments, the therapeutic agent can be actively loaded into the nano-VLP. For example, the nano-VLP can be exposed to conditions, such as electroporation, in which the viral envelope or viral membrane is made permeable to a solution containing therapeutic agent thereby allowing for the therapeutic agent to enter into the internal volume of the nano-VLP.

The moieties that may be encapsulated in the nano-VLP include therapeutic and diagnostic moieties, e.g., nucleic acid sequences, radionuclides, hormones, peptides, antiviral agents, antitumor agents, cell growth modulating agents, cell growth inhibitors, cytokines, antigens, toxins, adjuvants, etc.

The moiety encapsulated should not adversely affect the nano-VLP, or nano-VLP stability. This may be determined by producing nano-VLP containing the desired moiety and assessing its effects, if any, on nano-VLP stability.

The subject nano-VLP, which contain a desired moiety, upon administration to a desired host, should be taken up by cells normally infected by the particular filovirus, e.g., epithelial cells, keratinocytes, etc. thereby providing for the potential internalization of said moiety into these cells. This may facilitate the use of subject nano-VLP for therapy because it enables the delivery of a therapeutic agent(s) into a desired cell, site, e.g., a cervical cancer site. This may provide a highly selective means of delivering desired therapies to target cells.

In case of DNAs or RNAs, the encapsulated nucleic acid sequence can be up to 19 kilobases, the size of the particular filovirus. However, typically, the encapsulated sequences will be smaller, e.g., on the order of 1-2 kilobases. Typically, the nucleic acids will encode a desired polypeptide, e.g., therapeutic, such as an enzyme, hormone, growth factor, etc. This sequence will further be operably linked to sequences that facilitate the expression thereof in the targeted host cells.

The nano-VLP of the present invention can be used to deliver any suitable cargo in a targeted or untargeted fashion.

Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. In some embodiments, the targeting component can be specific to only one target, such as a receptor. Suitable targets can include but are not limited to a nucleic acid, such as a DNA, RNA, or modified derivatives thereof. Suitable targets can also include but are not limited to a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell.

In certain embodiments, a targeting agent can include a target ligand, a small molecule mimic of a target ligand, or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

The therapeutic agent or agents used in the present invention can include any agent directed to treat a condition in a subject. In general, any therapeutic agent known in the art can be used, including without limitation agents listed in the United States Pharmacopeia (U.S.P.), Goodman and Gilman's The Pharmacological Basis of Therapeutics, 10.sup.th Ed., McGraw Hill, 2001; Katzung, Ed., Basic and Clinical Pharmacology, McGraw-Hill/Appleton & Lange, 8.sup.th ed., Sep. 21, 2000; Physician's Desk Reference (Thomson Publishing; and/or The Merck Manual of Diagnosis and Therapy, 18.sup.th ed., 2006, Beers and Berkow, Eds., Merck Publishing Group; or, in the case of animals, The Merck Veterinary Manual, 9.sup.th ed., Kahn Ed., Merck Publishing Group, 2005; all of which are incorporated herein by reference.

Therapeutic agents can be selected depending on the type of disease desired to be treated. For example, certain types of cancers or tumors, such as carcinoma, sarcoma, leukemia, lymphoma, myeloma, and central nervous system cancers as well as solid tumors and mixed tumors, can involve administration of the same or possibly different therapeutic agents. In certain embodiments, a therapeutic agent can be delivered to treat or affect a cancerous condition in a subject and can include chemotherapeutic agents, such as alkylating agents, antimetabolites, anthracyclines, alkaloids, topoisomerase inhibitors, and other anticancer agents. In some embodiments, the agents can include antisense agents, microRNA, siRNA and/or shRNA agents.

Therapeutic agents can include an anticancer agent or cytotoxic agent including but not limited to avastin, doxorubicin, temzolomide, rapamycin, platins such as cisplatin, oxaliplatin and carboplatin, cytidines, azacytidines, 5-fluorouracil (5-FU), gemcitabine, capecitabine, camptothecin, bleomycin, daunorubicin, vincristine, topotecane or taxanes, such as paclitaxel and docetaxel.

Therapeutic agents of the present invention can also include radionuclides for use in therapeutic applications. For example, emitters of Auger electrons, such as ¹¹¹In, can be combined with a chelate, such as diethylenetriaminepentaacetic acid (DTPA) or 1,4,7,10-tetraazacyclododecane-1,4,7,10-tetraacetic acid (DOTA), and included in a nanoparticle to be used for treatment. Other suitable radionuclide and/or radionuclide-chelate combinations can include but are not limited to beta radionuclides (¹⁷⁷Lu, ¹⁵³Sm, ^{88/90}Y) with DOTA, ⁶⁴Cu-TETA, ^{1188/186}Re (CO)₃-IDA; ^{1188/186}Re (CO) triamines (cyclic or linear), ^{1188/186}Re(CO)₃-Enpy2, and ^{1188/186}Re(CO)₃-DTPA.

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (about 700-900 nm, e.g., indocyanines) are preferred. For topical visualization using an endoscopic method, any dyes absorbing in the visible range are suitable.

In yet other embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5th Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to gadopentetic acid, gadoteric acid, gadodiamide, gadolinium, gadoteridol, mangafodipir, gadoversetamide, ferric ammonium citrate, gadobenic acid, gadobutrol, or gadoxetic acid. Superparamagnetic agents can include but are not limited to superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexyl, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexyl, iopentol, ioversol, iobitridol, iodixanol, iotrolan and iosimenol.

nano-VLP of the present invention can also be used to deliver any expressed or expressible nucleic acid sequence to a cell for gene therapy or nucleic acid vaccination. The cells can be in vivo or in vitro during delivery. The nucleic acids can be any suitable nucleic acid, such as deoxyribonucleic acid (DNA) or ribonucleic acid (RNA). Moreover, any suitable cell can be used for delivery of the nucleic acids.

Gene therapy can be used to treat a variety of diseases, such as those caused by a single-gene defect or multiple-gene defects, by supplementing or altering genes within the host cell, thus treating the disease. Typically, gene therapy involves replacing a mutated gene, but can also include correcting a gene mutation or providing DNA encoding for a therapeutic protein. Gene therapy also includes delivery of a nucleic acid that binds to a particular messenger RNA (mRNA) produced by the mutant gene, effectively inactivating the mutant gene, also known as antisense therapy. Representative diseases that can be treated via gene and antisense therapy include, but are not limited to, cystic fibrosis, hemophilia, muscular dystrophy, sickle cell anemia, cancer, diabetes, amyotrophic lateral sclerosis (ALS), inflammatory diseases such as asthma and arthritis, and color blindness.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

When the nano-VLP are administered to deliver the cargo as described above, or as vaccine, the nano-VLP can be in any suitable composition with any suitable carrier, i.e., a physiologically acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline, water, buffered water, saline, glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17.sup.th ed., 1989).

Prior to administration, the nano-VLP compositions can be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized compositions.

The nano-VLP compositions can be made into aerosol formulations (i.e., they can be “nebulized”) to be administered via inhalation. In one embodiment, the lyophilized nVLP powder is used for aerosol administration. Aerosol formulations can be placed into pressurized acceptable propellants, such as dichlorodifluoromethane, propane, nitrogen, and the like.

Suitable formulations for rectal administration include, for example, suppositories, which includes an effective amount of a packaged composition with a suppository base. Suitable suppository bases include natural or synthetic triglycerides or paraffin hydrocarbons. In addition, it is also possible to use gelatin rectal capsules which contain a combination of the composition of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of nano-VLP compositions can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a nano-VLP composition. The unit dosage form can be a packaged preparation, the package containing discrete quantities of preparation. The composition can, if desired, also contain other compatible therapeutic agents.

Administration of the nano-VLP disclosed herein may be carried out by any suitable means, including both parenteral injection (such as intraperitoneal, subcutaneous, or intramuscular injection), by in ovo injection in birds, orally and by topical application of the nano-VLP (typically carried in the pharmaceutical formulation) to an airway surface. Topical application of the nano-VLP to an airway surface can be carried out by intranasal administration (e.g. by use of dropper, swab, or inhaler which deposits a pharmaceutical formulation intranasally). Topical application of the nano-VLP to an airway surface can also be carried out by inhalation administration, such as by creating respirable particles of a pharmaceutical formulation (including both solid particles and liquid particles) containing the lyophilized nano-VLP in powder form or as an aerosol suspension, and then causing the subject to inhale the respirable particles. Methods and apparatus for administering respirable particles of pharmaceutical formulations are well known, and any conventional technique can be employed.

A nano-VLP vaccine may be given in a single dose schedule, or preferably a multiple dose schedule in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. Examples of suitable immunization schedules include: (i) 0, 1 months and 6 months, (ii) 0, 7 days and 1 month, (iii) 0 and 1 month, (iv) 0 and 6 months, or other schedules sufficient to elicit the desired immune responses expected to confer protective immunity, or reduce disease symptoms, or reduce severity of disease.

In therapeutic use, the nano-VLP compositions including a therapeutic and/or diagnostic agent, as described above, can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, can be varied depending upon the requirements of the patient, the severity of the condition being treated, and the nano-VLP composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular nano-VLP composition in a particular patient. Determination of the proper dosage for a particular situation is within the skill of the practitioner. Generally, treatment is initiated with smaller dosages which are less than the optimum dose of the nano-VLP composition. Thereafter, the dosage is increased by small increments until the optimum effect under circumstances is reached. For convenience, the total daily dosage can be divided and administered in portions during the day, if desired.

In a further embodiment, the present invention relates to a method of detecting the presence of antibodies against Ebola virus or Marburg virus in a sample. When a hybrid, multi-immunogen nano-VLP is used, detection of the source of the foreign antigen is also possible. Using standard methodology well known in the art, a diagnostic assay can be constructed by coating on a surface (i.e. a solid support for example, a microtitration plate, a membrane (e.g. nitrocellulose membrane) or a dipstick, all or a unique portion of any of the Ebola or Marburg nano-VLP described above, and contacting it with the serum of a person or animal suspected of having an infection. The presence of a resulting complex formed between the nano-VLP and serum antibodies specific therefor can be detected by any of the known methods common in the art, such as fluorescent antibody spectroscopy or colorimetry. This method of detection can be used, for example, for the diagnosis of Ebola or Marburg infection, presence of antibodies to the pathogen from which the immunogen in the multi-immunogen nano-VLP was derived, and for determining the degree to which an individual has developed virus-specific Abs after administration of a vaccine.

In another embodiment, the present invention relates to a diagnostic kit which contains the nano-VLP described above and ancillary reagents that are well known in the art and that are suitable for use in detecting the presence of antibodies to Ebola or Marburg in serum or a tissue sample. Tissue samples contemplated can be from monkeys, humans, or other mammals.

The present invention also provides kits which are useful for carrying out the present invention. The present kits comprise a first container means containing the above-described nano-VLP. The kit also comprises other container means containing solutions necessary or convenient for carrying out the invention. The container means can be made of glass, plastic or foil and can be a vial, bottle, pouch, tube, bag, etc. The kit may also contain written information, such as procedures for carrying out the present invention or analytical information, such as the amount of reagent contained in the first container means. The container means may be in another container means, e.g. a box or a bag, along with the written information.

The following examples are included to demonstrate preferred embodiments of the invention. It should be appreciated by those of skill in the art that the techniques disclosed in the examples which follow represent techniques discovered by the inventors and thought to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All documents cited herein are hereby incorporated in their entirety by reference thereto.

The following materials and methods were used in the Examples below.

VLP Production and Purification

VLPs were produced at NCI and USAMRIID using a modification of the procedure described by Warfield et al. [Warfield et al., 2003, supra]. In brief, VLPs were created by transfecting HEK 293 cells with expression vectors containing the genes for GP and VP40 proteins. After a low-speed centrifugation to remove cells, VLP in the culture supernatant were centrifuged to a pellet using a Beckman JLA10.5 rotor at 10,000 rpm (˜18,500×g). The pellet was resuspended in PBS and applied to a 10-60% sucrose gradient. The gradient was then spun at 174,000×g for 14 hours. The resulting band was removed from the gradient and washed twice in sterile PBS. The VLP were resuspended in PBS and stored at 4° C., to be used in freeze/thaw studies. VLP were also produced under a contract at Paragon Bioservices, using a sucrose-gradient based method performed at a larger scale with a Wave bioreactor and HEK 293F suspension cells. These samples were stored frozen at −80° C.

The concentration of GP in the VLP was determined at Paragon by quantitative Western blotting with antibody 6D8, using recombinant soluble GP protein with a C-terminal His-tag in place of the transmembrane peptide as the standard. Subsequent measurement of GP concentrations of VLP samples were done by ELISA at USAMRIID and gave results within 10% of those found by Western blotting. GP was typically 20-30% of the total protein as measured by bicinchoninic acid assay. To measure GP concentration by ELISA, a standard curve was prepared by adhering recombinant soluble GP protein overnight to an ELISA plate (Immulon 2HB from Thermo Fisher #3455, Waltham, Mass.) in carbonate buffer at pH 9.5 (Data not shown). The soluble GP protein was expressed in HEK293 c18 cells, and had the transmembrane region replaced by a His-tag for purification. It was quantitated by UV absorbance (A₂₈₀ 1 mg/mL=1.30). GP on the ELISA plate was detected by probing with antibody 6D8 and an HRP-conjugated goat anti-mouse secondary antibody (Thermo Fisher #31430, Waltham, Mass.). The absorbance at 408 nm was fit to a 4-parameter logistic equation using SigmaPlot.

### Sonication and Filtration

Sonication of VLP was done using a Branson Sonifier 250 with a ⅛ inch tapered microtip. To prepare VLP samples for further study, 0.5 mL of VLP in PBS were sonicated for 3 sets of 3 pulses of 1 sec duration, at 50% duty cycle with the output control at 5.5. Samples were chilled on ice after each set of pulses. Filtration was done by passage through a 2.5 cm Supor syringe filter (Pall, Port Washington, N.Y.) of either 0.45 μm or 0.8/0.2 μm pore size.

### Electron Microscopy

Samples of VLP were adsorbed to formvar/carbon coated grids for electron microscopy and stained with PTA (phosphotungstic acid) or uranyl acetate. Samples were evaluated on a JEOL 1011 transmission electron microscope at 80 kV and digital images were acquired using an Advanced Microscopy Techniques (Danvers, Mass.) digital camera system.

### Particle Sizing

The particle size distribution of the samples was measured using an IZON qViro (Cambridge, Mass.) scanning ion occlusion sensing device. An IZON NP200A pore (nominal 200 nm diameter) was used, which was stretched until particles flowed freely. The voltage was 0.3-0.4 V and the pressure was varied up to 1.5 kPa. The sample introduced into the upper chamber contained 40 μL of VLP with 0.4-0.8 μg/mL [GP]. The buffer was PBS with 50 μg/mL Tween-80. Between 800 and 1500 data points were collected for each experiment. The calibration standard was 217 nm polystyrene beads (SKP200B) diluted to 1×10⁹ particles/mL concentration.

Particle concentrations were determined from the pressure dependence of the event rate (Roberts et al., 2012, Biosens Bioelectron 31:17-25). The concentration of an unknown sample (C₂) was found from the slope of the pressure dependence of the sample's event rate (g₂), the calibration standard's slope (g₁), and the concentration of the standard (C₁):

$${C}_{2} = ( \frac{{g}_{2}}{{g}_{1}} ) {C}_{1}$$

### Conformational ELISA

VLP samples were diluted in 0.2 M sodium carbonate/bicarbonate buffer at pH 9.5 for coating of an Immulon 2HB plate (Thermo Fisher, Waltham, Mass.) overnight at 4° C. After washing four times with PBS/0.05% Tween-20, plates were blocked with 3% dry milk in PBS for 1.5 h at 37° C. The wells were probed with antibody 6D8, 6D3, or 13C6 in 0.2 casein/PBS. 6D8 was used at 2.5 μg/mL, 6D3 at 1 μg/mL, and 13C6 at 1.6 μg/mL. HRP-labeled goat anti-mouse-Fc (Thermo Fisher, Waltham, Mass.) was the secondary antibody. Plates were developed with an ABTS peroxidase substrate (KPL), stopped with 1% SDS, and the absorbance read at 408 nm.

### Nano-VLP Purification

Transfection of HEK 293 c18 cells (ATCC CRL-10852) with expression vectors for Ebola Zaire GP and VP40 was done using polyethylenimine (PEI) as transfectant and 500 mL shaker flask cultures. After three days growth, the culture was centrifuged to remove cells (1773×g for 15 min). The supernatant was then centrifuged at higher speed to pellet the VLP (18.6K×g for 2 h). The pellet from each liter of cells was resuspended in 10 mL of 10 mM sodium phosphate, 50 mM NaCl, pH 7.4 (buffer 1), and kept on ice. The resuspended pellet was sonicated to increase the fluidity of the sample and decrease the length of filamentous VLP, allowing for more efficient filtration.

Sonication of VLP was done using a Branson Sonifier 250 (Danbury, Conn.) as described above. 10-12 mL of crude VLP in a 50 mL conical tube were sonicated for 4 sets of 3 pulses of 1 sec duration, at 50% duty cycle with the output control at 5.5. Samples were chilled on ice after each set of pulses. After sonication, samples were diluted with an equal volume of buffer 1. The sample was then filtered once through a 2.5 cm GF/D filter (Whatman glass microfiber; GE Healthcare Life Sciences, Piscatawy, N.J.), and then twice through GF/F filters. These steps removed a quantity of yellow colloidal material. 20 mL of the filtered sample was then run over a Sartorius Sartobind S-75 membrane chromatography disc (Sartorius, Frankfurt, Germany), pre-equilibrated with buffer 1 on a GE Healthcare FPLC system. The VLP flowed through the S-75 disc. Bound contaminants were eluted for analysis only using a gradient to 2 M NaCl. The flow rate was 1 mL/min at a backpressure of 0.16 MPa. S-75 discs were not reused.

The flow-through of the S-75 disc was again sonicated for 3 sets of 3 pulses to reduce particle size. The sample was then centrifuged in 50 mL conical tubes for 2 h at 14.6K×g) in a swinging bucket rotor. The pellet was resuspended in 2 mL of a sterile 0.5×PBS solution with 5% trehalose. The final filtration step to remove aggregates was done with a 2.5 cm Pall (Port Washington, N.Y.) 0.45 μM Supor filter. Bioburden was tested by plating on LB agar at 37° C. and no growth was found. Use of a 0.2 μM filter caused additional loss of the VLP and was not necessary to remove microbes for laboratory reagent purposes. Samples were then either stored frozen at −80° C., or lyophilized. Samples for SDS-PAGE were reduced and heated before loading on NuPage 4-12% Bis-Tris gels (Life Technologies, Carlsbad, Calif.).

### Lyophilization

For lyophilization, 262 μL volume samples in 0.5×PBS+5% trehalose containing 60 μg GP each were placed in 2 mL glass vials. 5% trehalose was included in the buffer as a protectant against stresses associated with freezing and dehydration. The samples were frozen on dry ice, and then placed on the shelf of a VirTis AdVantage ES freeze-dryer at −20° C. for drying in one stage. After 24 h exposure to vacuum (80 MT), the samples were reduced to a white powder. For stress-testing, the stoppered glass vial containing lyophilized VLP was placed in a 75° C. block for 1 h. A 26G needle was inserted to vent the cap during heating. All samples were resuspended in sterile water at the time of use.

### In Vivo Efficacy

VLP were diluted in sterile saline for intramuscular administration. When the adjuvant poly-ICLC was used, it was diluted with the VLP in sterile saline and co-administered. Lyophilized VLP were reconstituted to approximately 500 μg/ml in sterile water prior to dilution in sterile saline. For all vaccination studies, female C57BL/6 mice (age 8-10 weeks) were vaccinated intramuscularly two times with 100 μl of volume, with three weeks between vaccinations. Two weeks after the final vaccination, peripheral blood was collected from vaccinated mice and antibody titers were measured using an IgG ELISA. Four weeks after the final vaccination, mice were challenged via the intraperitoneal (IP) route with 1,000 pfu of mouse adapted (ma)-EBOV (Bray et al., 1998, J Infect Dis 178:651-661). Research was conducted under an IACUC approved protocol in compliance with the Animal Welfare Act, PHS Policy, and other Federal statutes and regulations relating to animals and experiments involving animals. The facility where this research was conducted, USAMRIID, is accredited by the Association for Assessment and Accreditation of Laboratory Animal Care International, and it adheres to principles stated in the 8th Edition of the Guide for the Care and Use of Laboratory Animals, National Research Council, 2011.

### Anti-Glycoprotein ELISA

Anti-glycoprotein antibody titers were determined by ELISA. Two μg/mL of recombinant Ebola virus glycoprotein was incubated in a flat bottom 96 well plate overnight. Glycoprotein with a C-terminal His-tag and lacking the transmembrane was expressed in HEK 293 c18 cells and purified by IMAC. Plates were incubated with blocking buffer (5% milk, 0.05% Tween in PBS) for 2 hours, and then serum samples were added to plates. Samples were diluted by half log dilutions ranging from 2 to 5.5 logs. After 2 hours, plates were washed with PBS+0.05% Tween and goat anti-mouse IgG-HRP secondary antibody was added at a 0.6 μg/mL. One hour later, plates were washed and exposed using Sure Blue TMB 1-component substrate and stop solution (KPL), and the absorbance at 450 nm was recorded. Serum from unvaccinated animals was used to establish background and titers were defined as the serum dilution resulting in an absorbance greater than 0.2, where background was universally less than 0.2. Serum from animals previously determined to contain anti-glycoprotein antibody was included in each assay to serve as a positive control.

### Example 1

### Impact of Sonication and Filtration on the Size Distribution of VLP

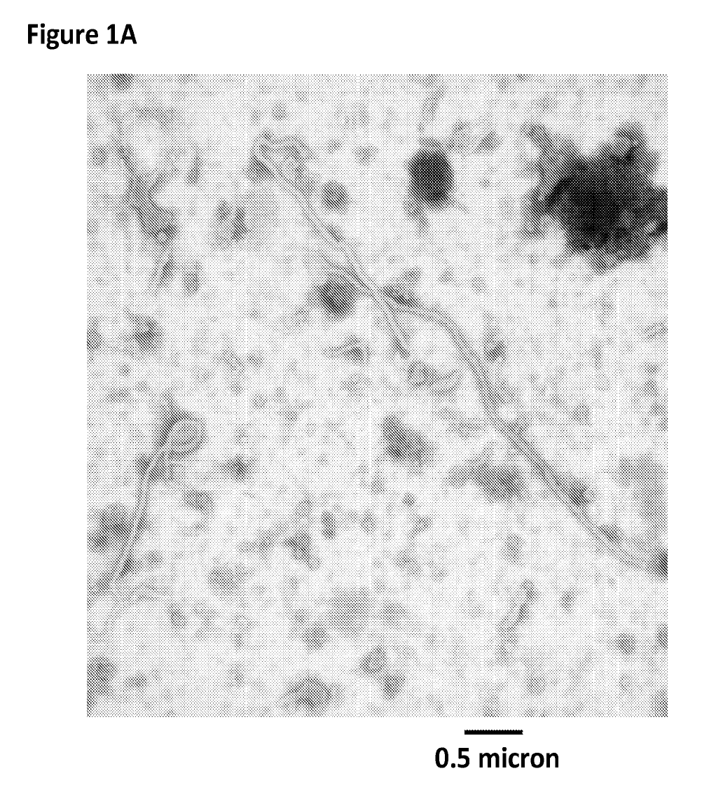

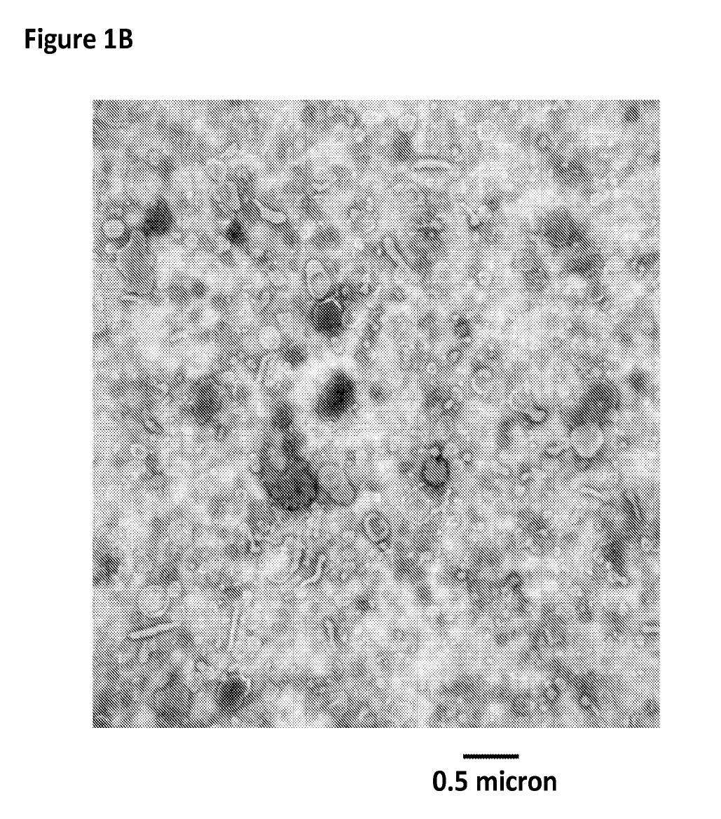

As seen in earlier analyses (Bavari et al., 2002, J Exp Med 195:593-602), electron microscopy revealed that the Ebola Zaire VLP samples contained long filaments (>1 mm) with bulbous regions that resemble Ebola virions, spherical particles similar to the head region of the virus, and some large irregular objects that might be cellular debris (FIG. 1A). Sonication of the VLP was optimized by varying the number of applied pulses until conditions were found that yielded fragments of up to ˜400 nm length (FIG. 1B.) Sonication for a total of nine pulses was adequate to fragment the VLP, but more extensive disruption for a total of 30 pulses resulted in substantial loss of the integrity of the VLP membrane. Filtration of the sonicated samples through either a 0.45 μm or 0.8/0.2 μm cutoff syringe filter yielded both spherical particles and fragments of filaments, and effectively removed the larger particles and aggregates. Sonication of VLP improved the yield of GP through a 0.8/0.2 mm filter from 10% to 50%, and through a 0.45 mm filter from 28% to 63%. The size distribution of the VLP samples was analyzed by Scanning Ion Occlusion Sensing (SIOS). In this method, particles in the sample are forced through a pore of adjustable size by an applied pressure and an electroosmotic potential. As particles pass through the pore, a change in current due to pore blockage is measured as a function of time. The amplitude of the decrease in current is proportional to the degree to which the pore is blocked, while the duration of the blockade event is the time required for the particle to transit the pore. One advantage of this method lies in the characterization of heterogeneous samples containing particles of different sizes and shapes, which may be detected as individual blockade events rather than averaged over the whole population.

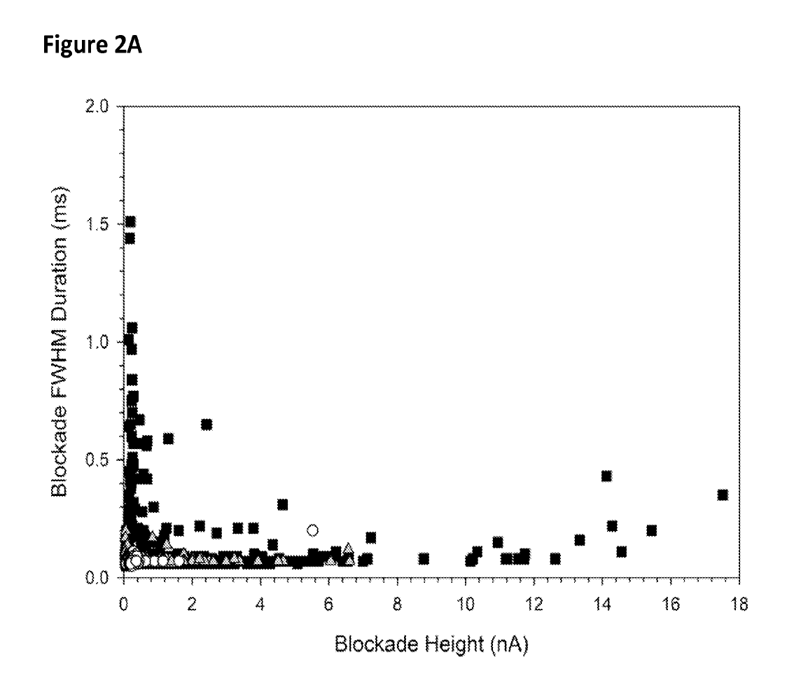

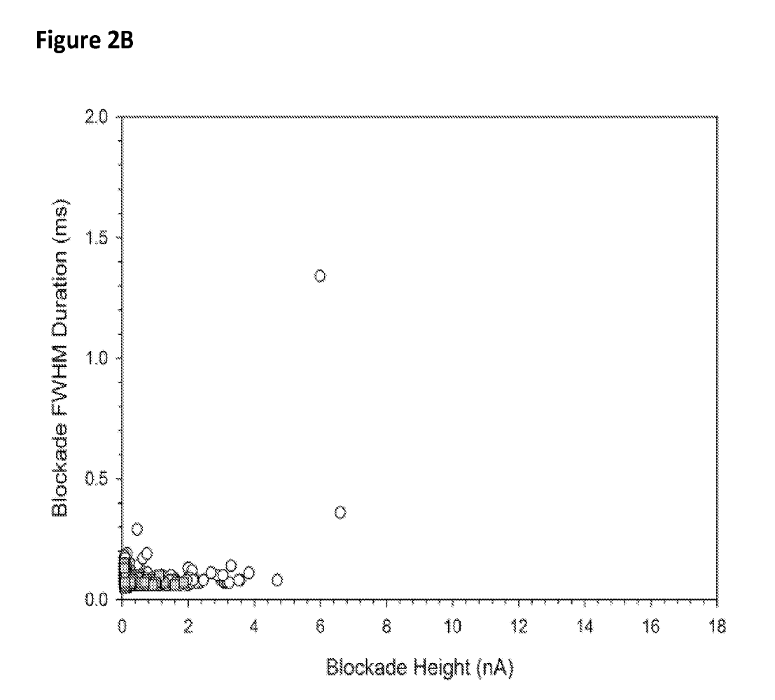

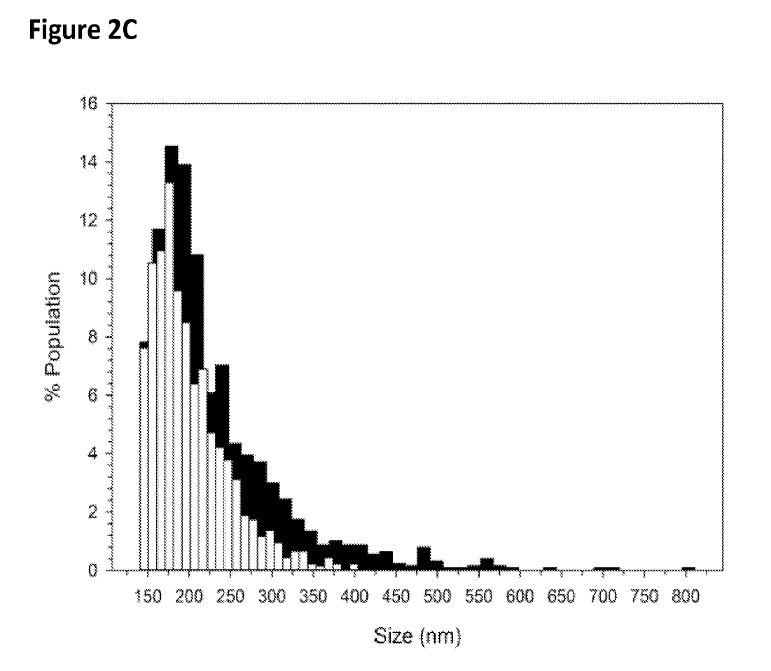

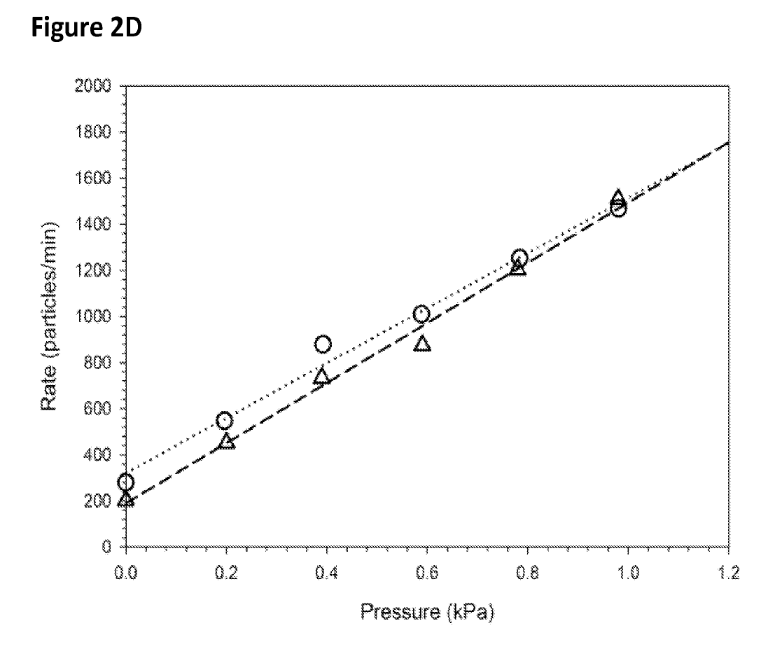

SIOS analysis of the undisrupted VLP yielded a bifurcated plot of the blockade event duration in ms (FWHM: full width at half maximum) vs. blockade height in nA (FIG. 2A). The events of lesser height and longer duration in the VLP sample are consistent with the transit of narrow, elongated filamentous particles through the pore. VLP filaments, which are 70-85 nm wide and up to 4 μm in length, may be expected to block the pore only partially, while taking a relatively long period of time to transit. In comparison, the spherical 217 nm polystyrene beads used for calibration showed a tight cluster of blockade events of less than 2 nA magnitude and 0.2 ms duration. Disruption of the VLP by sonication (Table 1) resulted in the loss of most events with greater blockade amplitudes (>2 nA) and nearly all those of longer duration (>0.2 ms), consistent with the disruption of filaments observed in the EM. Passage of the sonicated VLP through a 0.8/0.2 mm syringe filter removed the larger remaining particles (FIG. 2B).

TABLE 1
Nanopore analysis of VLP.

| | | | Mean |
|---|---|---|---|
| | % FWHM | % Height | particle |
| Sample | >0.2 ms | >2 nA | size (nm) |
| Undisrupted | 6.7 | 6.2 | |
| Sonicated | 0.1 | 1.9 | 227 |
| Sonicated and filtered 0.45 μm | 0.3 | 2.1 | 230 |
| Sonicated and filtered 0.8/0.2 μm | 0 | 0 | 202 |
| Incubated 37° C. 24 h | 2.7 | 11 | |
| Incubated 37° C. 96 h | 2.7 | 9.1 | |

FIG. 2C shows the calculated particle size distributions of the filtered samples, which were consistent with what was observed by EM. The mean particle size is normally calculated from SIOS data by calibration using beads of known size (Kozak et al., 2011, Nano Today 6:531-545). However, this analysis may not be appropriate when the sample contains a significant fraction of elongated, non-spherical particles. We therefore present mean particle sizes only for the sonicated samples (Table 1), which have a relatively narrow distribution of particle sizes and shapes. The smaller particles of less than 150 nm could not be simultaneously resolved in this nanopore measurement; however we have chosen to focus the particles >150 nm that were identifiable as VLP or fragments of VLP.

SIOS can also be used to determine particle concentrations from the rate of observed events compared to a calibration standard of known concentration (Roberts et al., 2012, supra). However, passage of intact VLP samples through the SIOS pore was frequently interrupted by blockages, presumably due to the larger aggregates present. These blockages did not affect the particle size measurements, which were determined from individual events. Experiments could be resumed by pipetting the sample up and down, but a consistent flow rate of particles through the pore could not be obtained. By removal of larger particles or aggregates from the sample, filtration allowed a smooth flow through the pore for more than 1 min.

Measurement of the rate of events at various applied pressures (FIG. 2D) was then done to allow calculation of the particle concentration from the relative slopes of the pressure dependence curves. For the filtered VLP sample shown, the concentration was 8.2±0.8×10¹¹ particles/mL (±SE, n=3).

### Thermostability of GP in VLP

GP only comprises approximately 20-30% of the total protein in VLP preparations, but it is the most significant component in terms of immunogenicity (Swenson et al., 2005, supra). The conformational integrity of GP is hypothesized to be important for immunogenicity. We therefore sought to develop an ELISA method that would characterize specifically the conformation of GP in VLP preparations subjected to applied stress. In this approach, we compared the relative amount of binding of antibodies that recognize either linear or conformational epitopes. Denaturation of GP would be expected to result in loss of recognition by the conformationally-sensitive antibodies, while binding of the linear epitope antibody would be maintained.

Antibody 6D8 recognizes a linear epitope of GP (a.a. 389-405), while 13C6 and 6D3 are conformationally-dependent (Lee and Saphire, 2009, Curent opinion in structural biology 19:408-417, Wilson et al., 2000, Science 287:1664-1666). 13C6 is known to bind to the glycan cap (Murin et al., 2014, Proc Natl Acad Sci USA 111:17182-17187). All are non-competing. We anticipated that if GP were denatured by applied stress, binding by 6D8 should remain relatively constant, while binding by 6D3 and 13C6 should decrease.

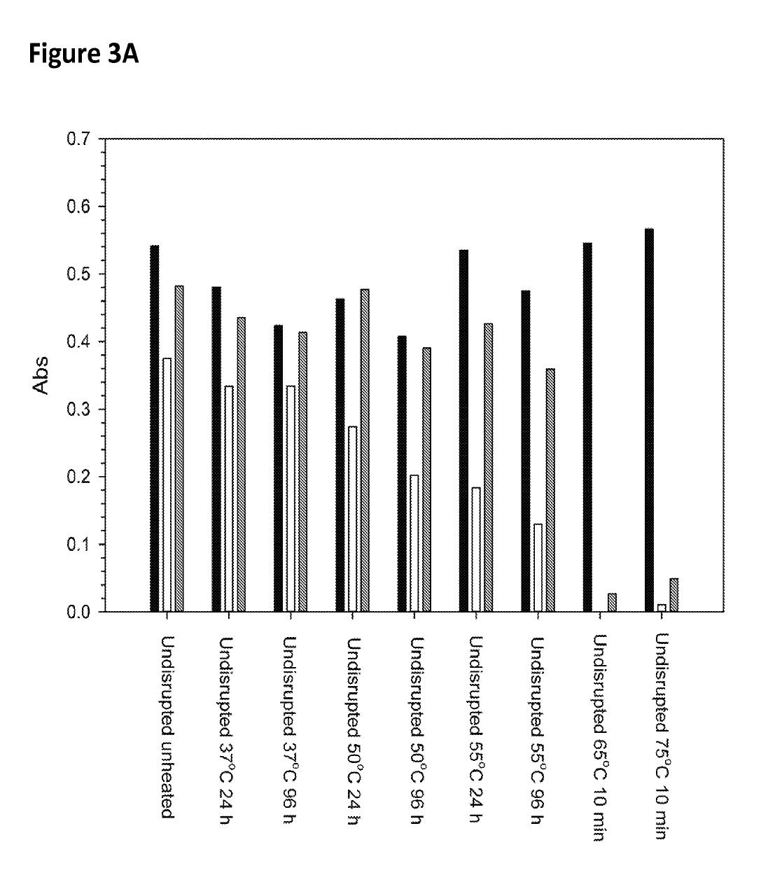

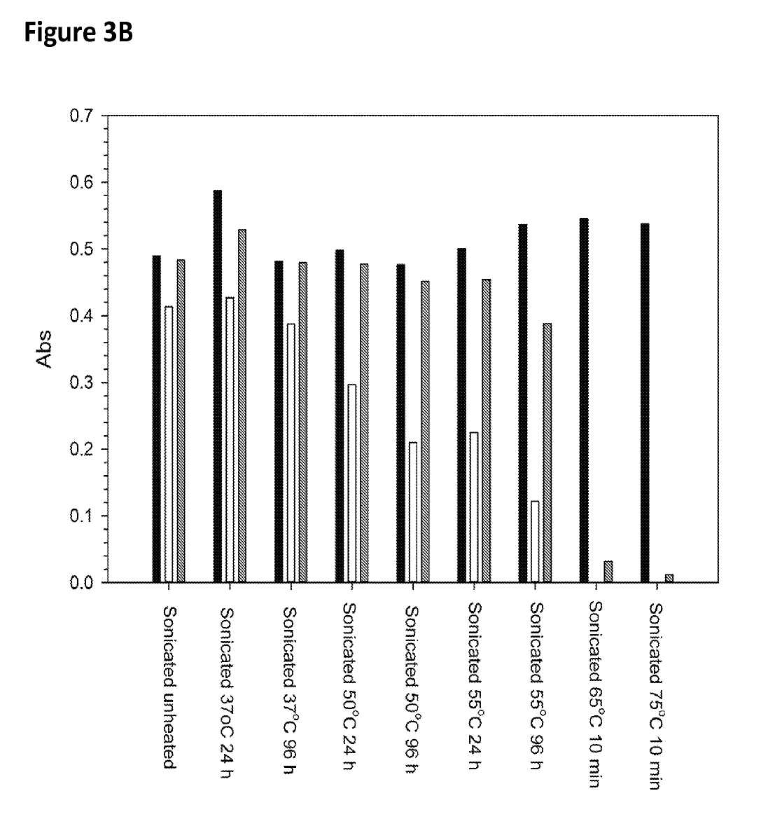

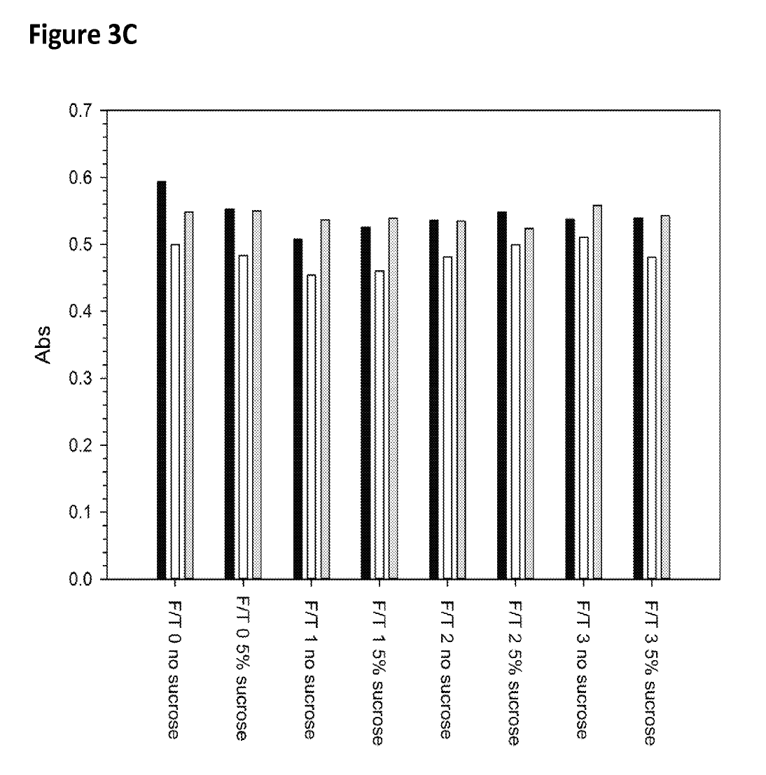

We used the ELISA method to test the resistance of both untreated, filamentous VLP and sonicated VLP to accelerated degradation by thermal stress. VLP samples were incubated at elevated temperatures for the indicated time periods in FIGS. 3A and 3B. Under all conditions tested, there was no difference between the impacts of thermal stress on sonicated VLP as compared to intact VLP, indicating that fragmentation of the VLP did not affect the conformational stability of GP.

Incubation at 37° C. for up to 96 h had no significant effect on the conformational integrity of GP, as judged by binding of the conformationally sensitive 6D3 and 13C6 antibodies. Binding of the control 6D8 was unchanged by incubation at the higher temperatures. At 50° C. or 55° C. for 24 h or 96 h, partial loss of 6D3 binding was observed, and after only 10 min at 65° C. or 75° C., 13C6 binding was also lost almost completely. The reason for the more rapid loss of 6D3 binding on heating is not yet clear, but could be due to partial denaturation of domains of the GP as temperature increased.

While incubation at 37° C. for as long as 96 h did not result in denaturation of GP, the integrity of filaments was affected. After incubating a VLP sample for 24 h at 37° C., we observed a decreased population of particles with long-duration pore transits (Table 1 and data not shown). This result indicates that longer filaments are not stable to extended incubation at 37° C., although GP conformation could be maintained even when filamentous structure was disrupted.

Having determined the sensitivity of GP in VLP to heating, we next evaluated the effects of repeated freeze/thawing on VLP particle size and the GP conformation. The VLP preparation used for these studies was purified at USAMRIID and tested after having never been frozen, or having been subjected to 1-3 rounds of freeze/thawing in PBS or PBS+5% sucrose. VLP were thawed in a 37° C. water bath and refrozen on dry ice. No significant change in GP conformation (FIG. 3C) as a result of freeze/thawing was found. Electron microscopy and particle sizing measurements also found no significant change in size as a result of freeze/thawing (data not shown).

### Immunopotency Testing

Based on our biochemical assessments, we had shown that optimized sonication can result in smaller, more homogenous VLP that expressed conformationally intact GP, while excessive heating of the VLP disrupted the GP conformation. To evaluate the relevance of these findings in terms of vaccine efficacy, we vaccinated C57BL/6 mice with VLP treated under the respective conditions and challenged them with mouse adapted-EBOV. Based on previous studies, we had shown that 10 μg of VLP, based on GP content, was the minimum sufficient vaccine dose to reliably achieve 90-100% protection from challenge (Martins et al. 2014, PLoS One 9:e89735). We therefore used this dose level as our baseline for analysis.

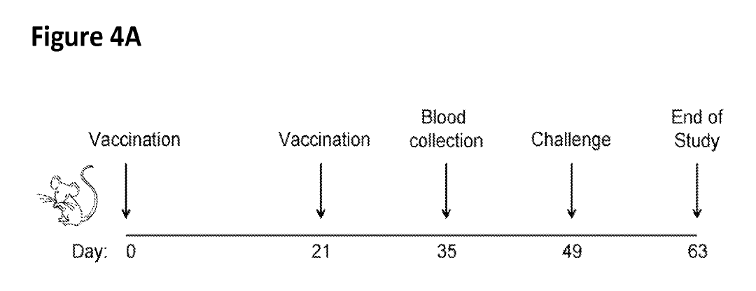

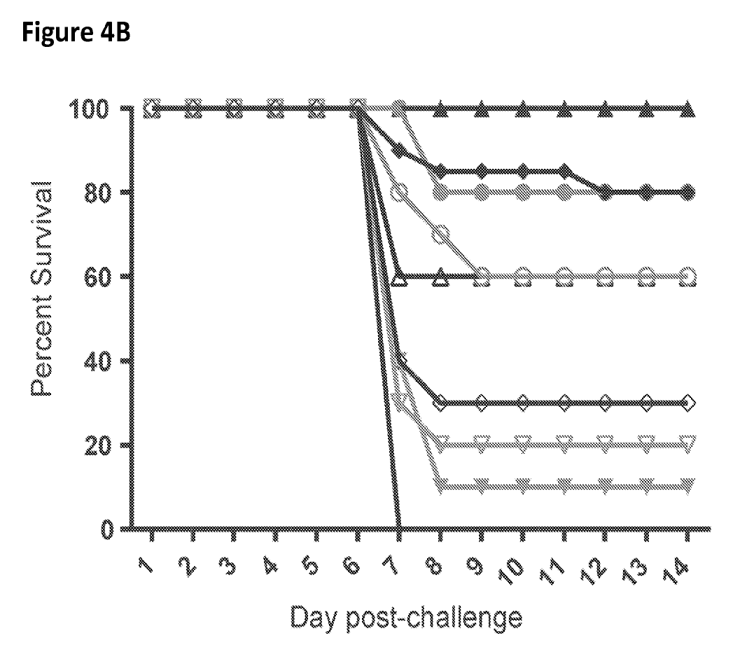

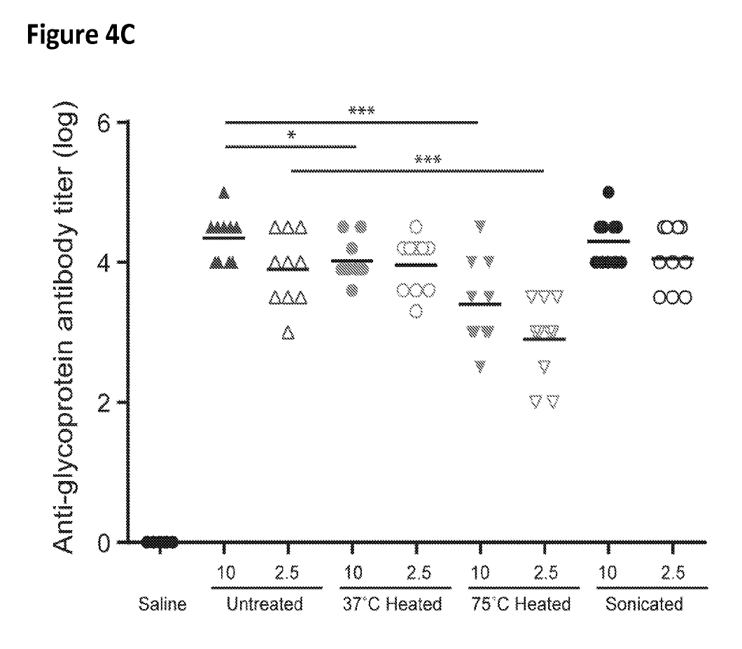

To evaluate the impacts of heating and sonication on VLP efficacy, we vaccinated animals with either 10 μg or 2.5 μg of VLP two times, on the schedule shown in FIG. 4A. As shown in FIG. 4 and Table 2 (experiment #1), VLP that were sonicated or heated at 37° C. for 96 h provided comparable protection to filamentous control VLP that were not treated, suggesting that these conditions did not impact immunogenicity. In contrast, VLP that were heated at 75° C. for 15 min did not provide protection from challenge, supporting a relationship between GP conformational integrity and immunogenicity. The p-values comparing survival of groups treated with control VLP or 75° C. heated VLP confirmed the deleterious effect of high temperature, while the effects of sonication or 37° C. incubation were not significant. Anti-GP antibody titers in animals vaccinated with VLP heated at 75° C. were also significantly lower than the titers achieved in animals vaccinated with undisrupted VLP, sonicated VLP, or VLP heated at 37° C., at both dose levels tested (FIG. 4).

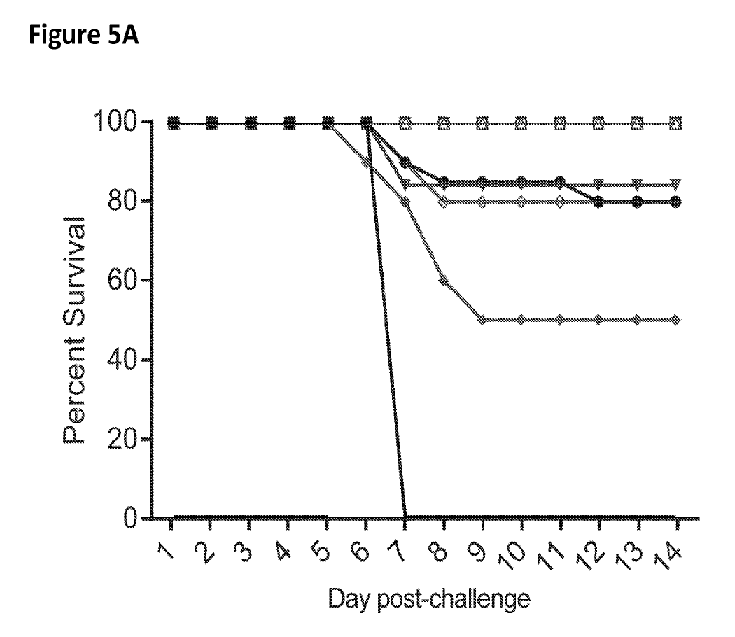

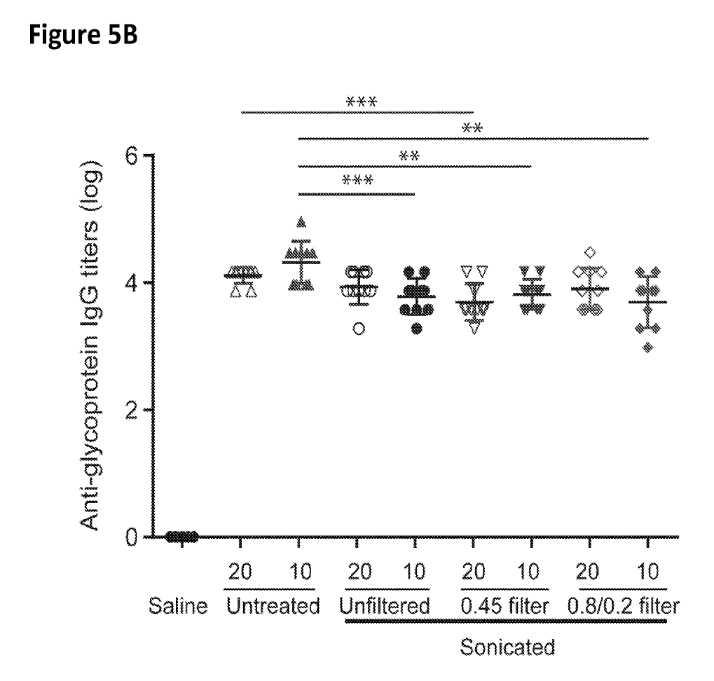

Considering that we had not observed 100% protection with the 10 μg dose level of sonicated VLP, we chose to vaccinate animals with 10 μg or 20 μg of VLP when comparing the impact of filtration on protection (FIG. 5 and Table 2, experiment #2). Sonicated VLP that were filtered through a 0.45 μm filter displayed comparable immunogenicity to those that were not filtered and to undisrupted VLP. VLP that were sonicated and then filtered with a 0.8/0.2 mm cutoff had slightly lower efficacy, which was statistically significant (p-value <0.05) at the 10 μg dose, though antibody titers were comparable between all groups.

TABLE 2
Survival of vaccinated mice after ma-Ebola challenge¹

| Experiment | Dose | Vaccination Materials | Survival | p-values² |
|---|---|---|---|---|
| 1 | 10 μg | Control VLP | 20/20 | |
| | | Heated, 37° C., 96 h | 8/10 | 0.1518 |
| | | Heated 75° C., 15 min | 1/10 | <.0001 |
| | | Sonicated | 16/20 | 0.3288 |
| | 2.5 μg | Control VLP | 6/10 | |
| | | Heated 37° C., 96 h | 6/10 | 0.9986 |
| | | Heated 75° C., 15 min | 2/10 | 0.1700 |
| | | Sonicated | 3/10 | 0.4040 |
| 2 | 10 μg | Control VLP | 20/20 | |
| | | Sonicated and filtered, | 16/19 | 0.3796 |
| | | 0.45 μm | | |
| | | Sonicated and filtered, | 5/10 | 0.0021 |
| | | 0.8/0.2 μm | | |
| | | Sonicated | 16/20 | 0.2418 |
| | 20 μg | Control VLP | 10/10 | |
| | | Sonicated and filtered, | 10/10 | 1.0000 |
| | | 0.45 μm | | |
| | | Sonicated and filtered, | 8/10 | 0.1050 |
| | | 0.8/0.2 μm | | |
| | | Sonicated | 10/10 | 1.0000 |

¹None of the animals vaccinated with saline survived challenge (n = 20).
²p-values were calculated using Fisher's exact tests to compare survival with Control VLP to each treatment group.

### Nano-VLP

The results above suggested to us that difficulties in manufacture of full-length filamentous VLP might be overcome by a reduction in their size, without loss of potency. We then developed a procedure for purification of nano-VLP consisting of a combination of centrifugation, sonication, glass fiber filtration, and Sartobind S-75 membrane negative capture steps; with a final filtration for removal of aggregates and bioburden. The SDS-gel electrophoresis pattern of the nano-VLP product and a preparation made by the sucrose-gradient procedure [Warfield et al., 2003, supra] show that the protein bands appear very similar in migration and relative amounts (data not shown). We also verified that the nano-VLP preparation possessed appropriate GP conformation by ELISA as done above (not shown). Measurement of the GP yield by ELISA with antibody 6D8 gave figures of 1.6-2.0 mg/L of cell culture, in comparison with ˜1 mg/mL for the sucrose gradient method. Both contained 20-30% GP when total protein was measured by BCA. A final 0.45 mm filtration provided adequate bioburden removal for laboratory usage, yielding a product that showed no bacterial growth on plating. Filtration with a 0.8/0.2 mm filter was possible but resulted in additional loss of product.

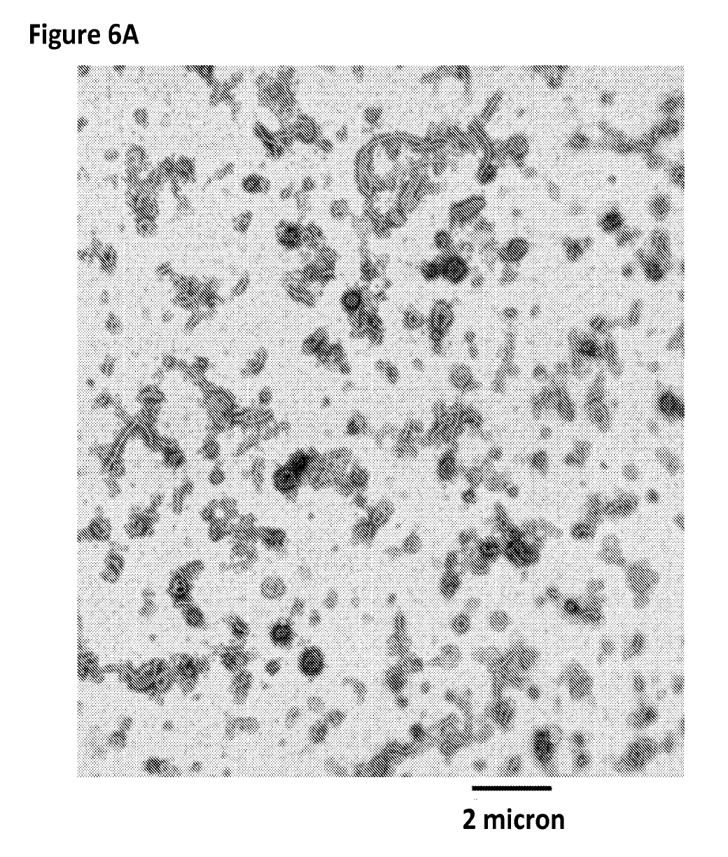

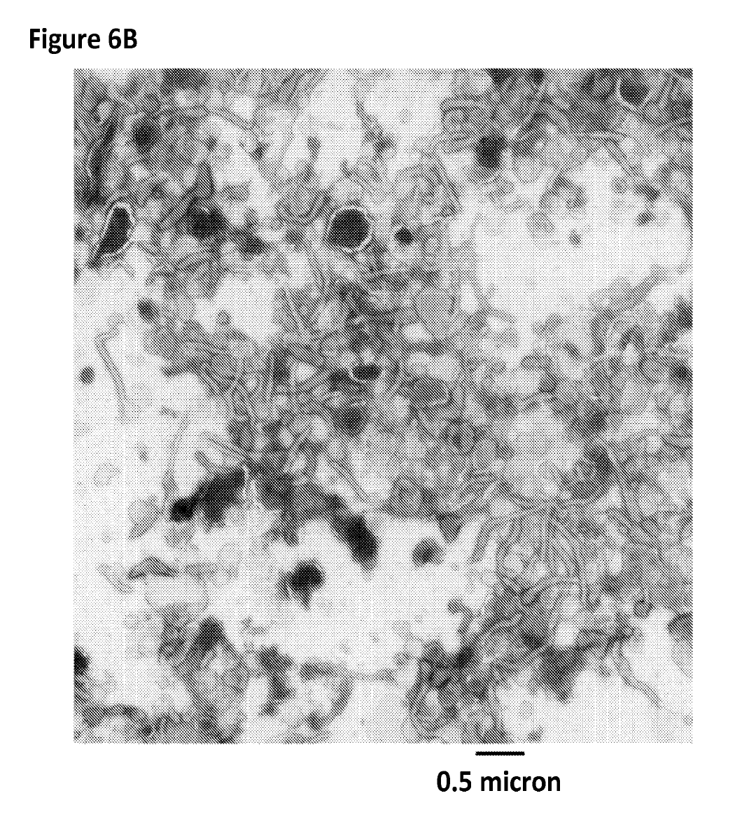

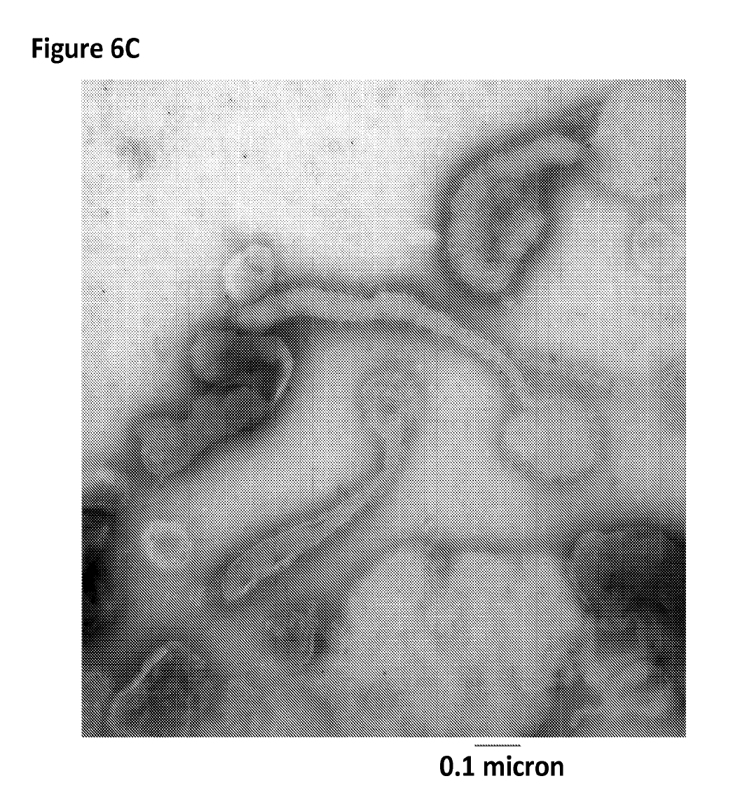

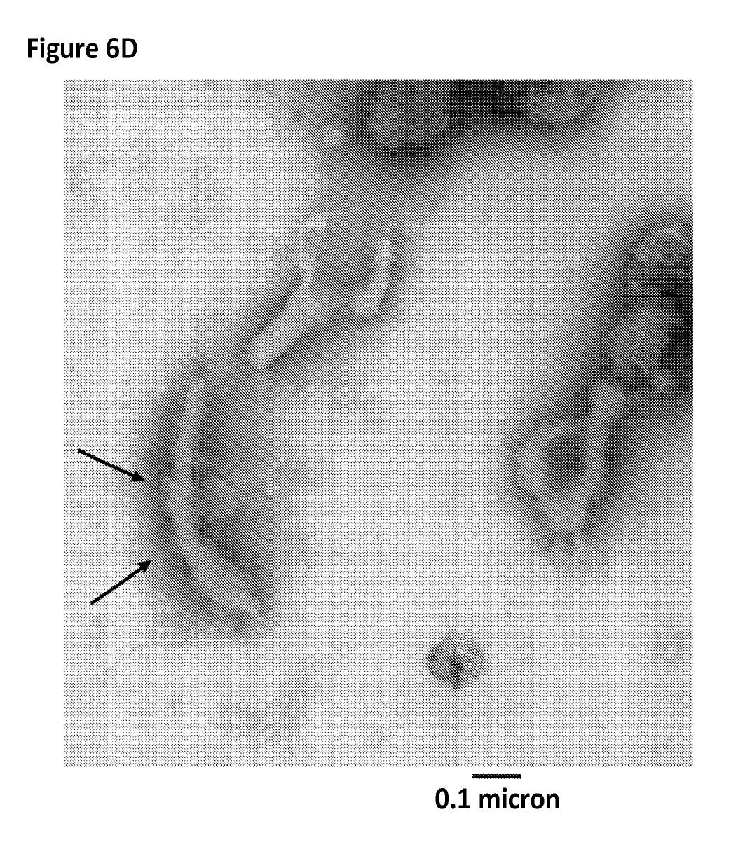

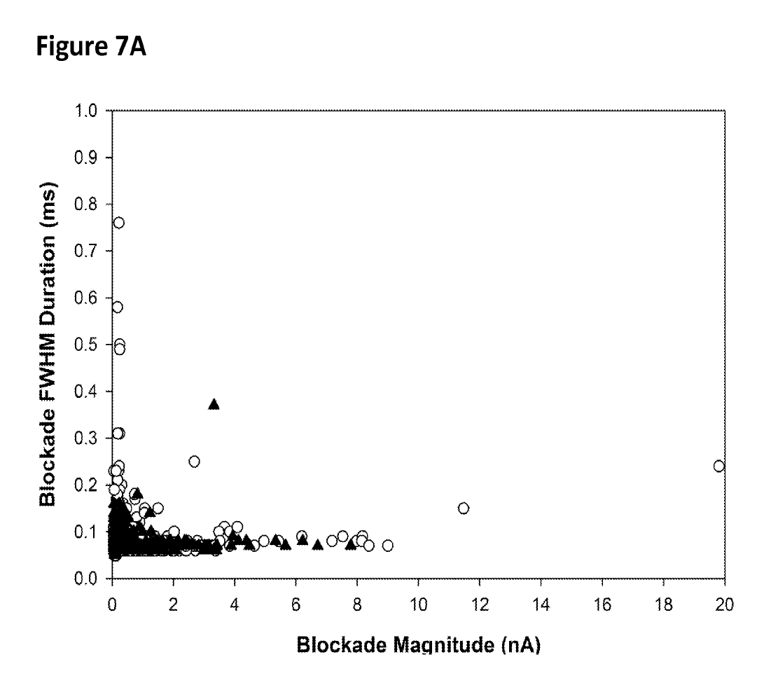

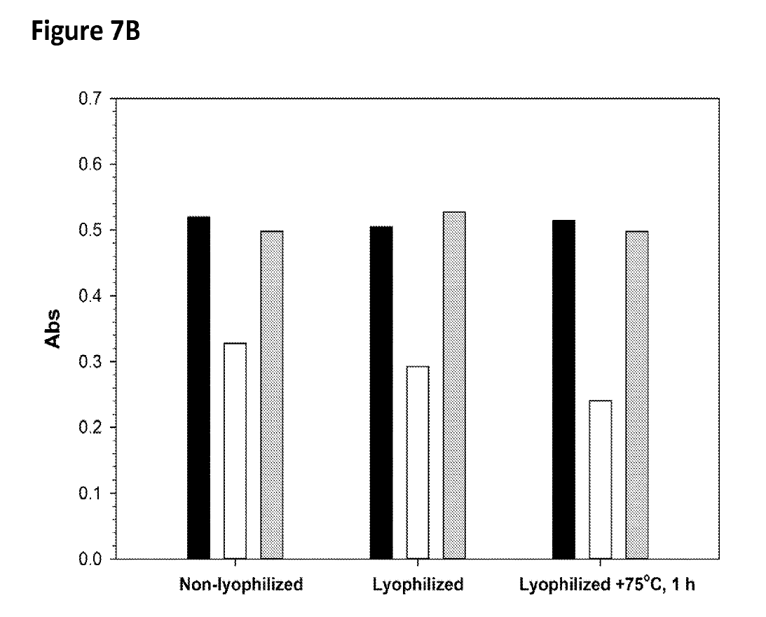

Transmission electron microscopy of the nano-VLP with PTA staining showed linear, branched, spherical and “6” shaped particles (FIG. 6A). Linear filaments were as long as 1.5 microns, but most particles were shorter filaments or spheres of ˜200 nm diameter. Particle size was also examined using a qViro device (FIG. 7A). With the nano-VLP sample, 7.1% of observed events had a passage duration of >0.2 ms. These events may represent passage of the shorter filaments found in FIG. 6A. Results from the qViro nanopore analysis appeared to be consistent with the EM data. The nano-VLP were not observed to possess lengths of several microns as is commonly found in sucrose-gradient purified VLP and authentic virions. The mean particle diameter was calculated to be 230 nm using a spherical bead standard; however, this method does not take into account the shape of the filament fragments. Although some of the fragments of filaments present were longer than the 0.45 micron pore size filter used in this preparation, their width was less than 100 nm.

We tested lyophilization of the nano-VLP as a possible means to formulate for stable storage. FIGS. 6B and 6C show electron micrographs of the lyophilized nano-VLP, after resuspension in water. The filaments retained structure after lyophilization, although some shortening of filaments occurred, which was confirmed by nanopore analysis (FIG. 7A). The calculated mean particle diameter of the lyophilized nano-VLP was 217 nm. The GP layer coating the lyophilized nano-VLP was visualized by negative staining in FIG. 6C, and was detected by immunogold-staining with antibody 6D8 (data not shown). Retention of the folded conformation of GP in the nano-VLP after lyophilization was confirmed using ELISA (FIG. 7B). Lyophilized VLP resuspended readily without the appearance of aggregation, and the particles flowed easily through the nanopore.

As described above, we observed that the GP of Ebola VLP underwent denaturation when heated in liquid suspension to 75° C. for 15 min, which was correlated with a nearly complete loss of protective capability in the mouse model. However, we found that lyophilized nano-VLP could be heated in the dry form before resuspension to 75° C. for at least 1 h, with little apparent loss of GP conformation as determined by conformational ELISA (FIG. 7B). Electron micrographs of the heated, lyophilized nano-VLP (FIG. 6D) showed that the filament fragments were relatively unchanged, although increased irregularities on the membrane surface were apparent.

### Efficacy as a Vaccine

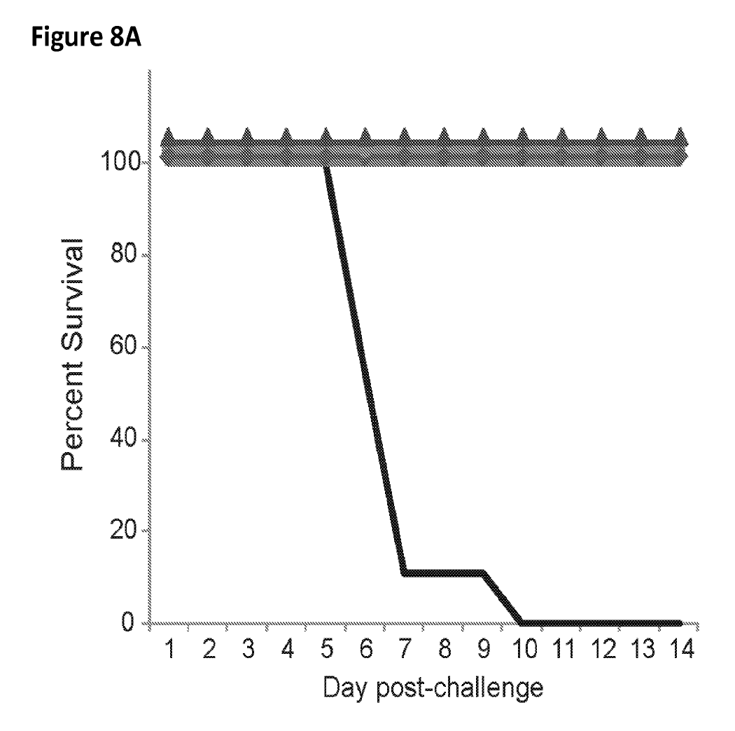

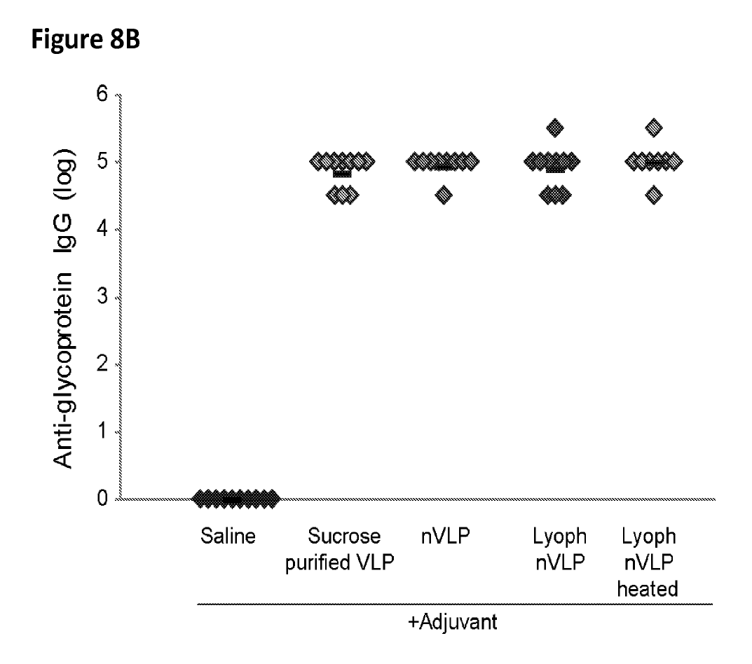

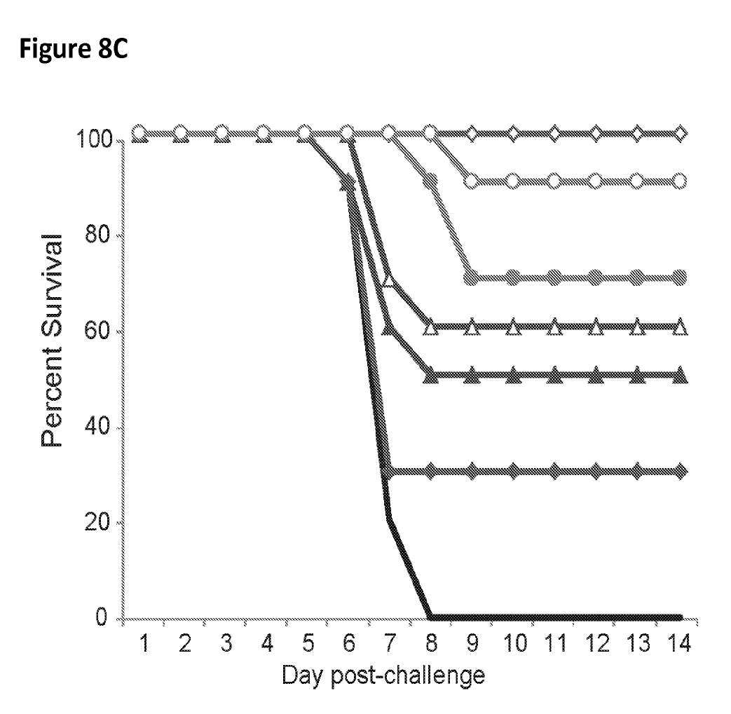

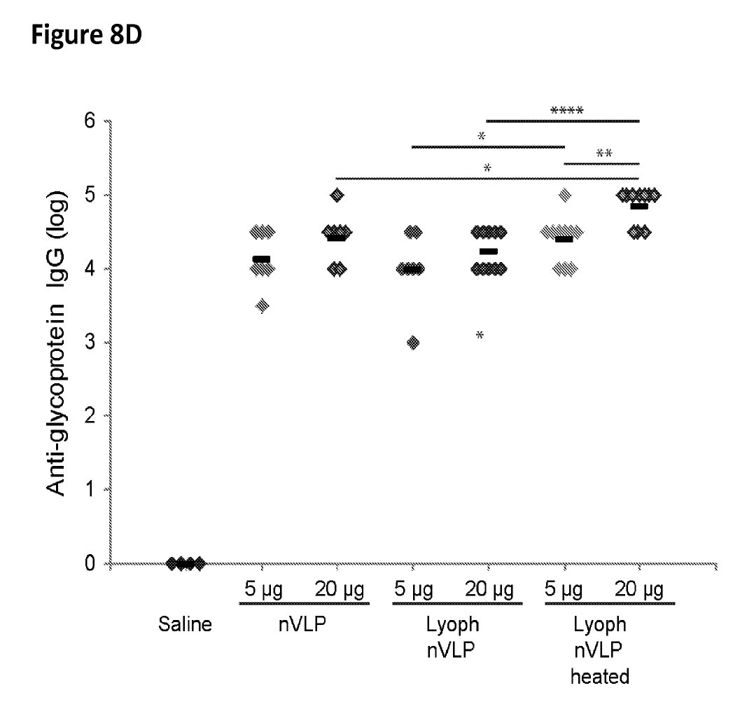

The ability of the nano-VLP to confer protection from mouse-adapted Ebola challenge was tested in a mouse assay. FIG. 8A shows results of an experiment in which mice were vaccinated with 5 μg doses of various VLP preparations [GP content of VLP] in combination with 10 μg poly-ICLC adjuvant. The VLP preparations that were tested are as follow: sucrose-gradient purified VLP, which served as a bridging control to previous studies; nano-VLP that were stored frozen; nano-VLP that were lyophilized; and lyophilized nano-VLP that had been heated in the vial at 75° C. for 1 h prior to resuspension. All (10/10) animals vaccinated with adjuvant only died after challenge with mouse-adapted Ebola virus, while all in the VLP-vaccinated groups survived (FIG. 8A). The anti-GP titers in the VLP-containing groups were uniformly high (FIG. 8B).

As a more stringent test of immunogenicity, we performed an experiment without the use of adjuvant (FIGS. 8C and 8D). nano-VLP were administered at doses of 5 or 20 μg GP content (Table 3). For each group, 20 μg conferred better protection than 5 μg. At the 20 μg dosage, nano-VLP that had been stored frozen yielded 6/10 survival, while lyophilized nano-VLP gave 10/10 survival and lyophilized/heated VLP gave 9/10. For all of these groups except lyophilized nano-VLP at 5 μg, survival vs. saline control was statistically significant. However, differences in survival between the vaccine groups, including the filamentous VLP used in earlier studies, were not statistically significant. Anti-GP titers also showed a dose response, and titers for the heated, lyophilized VLP were significantly higher than titers for lyophilized nano-VLP or frozen nano-VLP at the 20 μg dose level. The reason for this higher titer is not known.

TABLE 3
Survival of mice vaccinated with nano-VLP without adjuvant¹

| Dose | Vaccination Materials | Survival | p-values² |
|---|---|---|---|
| 20 μg | nano-VLP | 6/10 | 0.0029 |
| | Lyophilized nano-VLP | 10/10 | <.0001 |
| | Lyophilized and heated nano-VLP | 9/10 | <.0001 |
| 5 μg | nano-VLP | 5/10 | 0.0145 |
| | Lyophilized nano-VLP | 3/10 | 0.1463 |
| | Lyophilized and heated nano-VLP | 7/10 | 0.0004 |

¹None of the animals vaccinated with saline survived challenge (n = 10).
²p-values compare survival of each treatment group vs. saline.

### DISCUSSION

### VLP Stability and GP Conformation

Vaccines that can withstand ambient temperatures for extended periods are highly desirable to avoid loss of potency if the cold chain is broken. Thermal stability studies on VLP indicated that the GP antigen can withstand moderately high temperature stress for an extended period. Incubation at 55° C. for more than 24 h resulted in partial loss of conformation, while at 75° C. denaturation occurred rapidly as measured by ELISA. No significant loss of GP conformation was observed with 96 h incubation at 37° C., however. Loss of conformational integrity in the ELISA was correlated to loss of immunopotency in a mouse bioassay, demonstrating that the conformational ELISA method introduced herein can be used to predict rapidly the decreased potency of thermally stressed Ebola VLP vaccine samples. Structural studies of neutralizing antibodies to Ebola GP support the importance of GP conformation in antibody recognition (Lee et al., 2008, Nature 454:177-182).

Only one other study has examined filovirus VLP stability systematically, to our knowledge (Hu et al., 2011, J Pharm Sci 100:5156-5173). Those authors used biophysical methods on VLP derived from a baculovirus expression system, while we employed a human cell-based system and developed approaches to focus specifically on antigenic integrity. The light scattering and circular dichroism measurements of Hu et al. indicated that at neutral pH Ebola VLP's lost conformational integrity above 50-60° C. Although the thermal transitions were broad, and global properties of the VLP were assayed rather than those of GP specifically, those results are consistent with ours.

Particle Size Vs. Immunogenicity

Previous studies have found that particles greater 500 nm in size are processed more slowly by the immune system due to exclusion from direct drainage to lymph nodes (Bachmann and Jennings 2010, supra). This fact might suggest that large filamentous VLP would be less immunogenic than smaller VLP. However, we found no significant difference in potency between the filamentous and sonicated VLP using the mouse vaccination model. The roughly 230 nm average size of the sonicated VLP was large enough to provide the repeated antigen array necessary for strong stimulation of the immune response by GP (Wahl-Jensen et al., 2005, supra). Passage of the sonicated VLP through a 0.45 μm filter did not appear to affect potency, but passage through a 0.8/0.2 μm filter resulted in a lower survival rate after challenge. However, a statistically significant difference in potency of the more stringently filtered VLP was observed only at the 10 μg dose (Table 2).

To the extent that the mouse model is predictive, our results indicate that the presence of intact filaments is not essential to a VLP vaccine. The finding that reduction of particle size by sonication did not greatly change immunogenicity may be due at least in part to disruption of longer VLP filaments in vivo. We observed that extended incubation at 37° C. resulted in shortening of filaments, as determined by nanopore sizing measurements. This suggests that VLP filaments will breakdown into pieces after injection into the body of an animal, with the result being that the large initial size of the VLP may not be an impediment to access to the lymphatic system.

### Nano-VLP and Progress in Vaccine Development

We have described a new process for purification of a filovirus VLP vaccine that is more rapid and easily scaled for manufacturing than the previous sucrose gradient-based method. The size of the nano-VLP was smaller than that of complete Ebola virions, with spherical particles of roughly 230 nm diameter, and filaments of around 500 nm length. Nevertheless, the Ebola nano-VLP remained larger than typical viruses and GP in the native conformation was present on the surface of the nano-VLP. Reduction in size of the VLP facilitated their purification by membrane chromatography, which is more easily adapted to large-scale production than a sucrose gradient method. The resulting product was also more uniform in size, although differences in morphology between spherical and filamentous fragments remained. Further advancements will be necessary to bring our laboratory-scale method to cGMP scale and meet all FDA criteria for product licensure.

We have also presented assays for in vitro testing of GP conformational integrity in the context of VLP, the presence and relative population of filaments, and the concentration of particles in filtered VLP samples. The observed correlation of the conformational ELISA with the mouse immunopotency assay may allow its use in the future as a more rapid quality-control assay for the VLP and other types of Ebola vaccines, while the concentration of particles can be measured using the rate of events observed during flow through a nanopore.

Our results indicated that the lyophilized nano-VLP preparation was highly thermostable, suggesting that long-term storage of the lyophilized formulation without refrigeration is possible. Elimination of a cold chain requirement would decrease costs associated with the vaccine and greatly ease distribution to locales without reliable electricity, especially important for a tropical disease (Chen and Kristensen, 2009, Expert Rev Vaccines 8:547-557).

1-16. (canceled)

17: A nano-virus-like particle (VLP) composition.

18: The composition of claim 17 wherein said nano-VLP is Ebola or Marburg.

19: The composition of claim 18 wherein the nano-VLP is spherical.

20: The composition of claim 18 wherein the nano-VLP is filamentous.

21: The composition of claim 19 wherein the spherical nano-VLP diameter is from about 100 nm to about 400 nm.

22: The composition of claim 20 wherein the filamentous nano-VLP is from about 300 nm in length to about 1000 nm in length.

23: A filovirus vaccine comprising the composition of claim 18.

24: The composition of claim 18 wherein the composition is a lyophilized powder.

25: A vaccine comprising the lyophilized powder of claim 28.

26: An immunological composition comprising the composition of claim 18.

27: An immunological composition comprising the composition of claim 24.

28: A method for preparing purified nano-VLP from intact VLP comprising:
(i) isolating intact VLP from cells transfected with at least filovirus glycoprotein (GP) and VP40,
(ii) deaggregating the VLP to produce nano-VLP, and
(iii) purifying the nano-VLP.

29: The method of claim 28 wherein the deaggregation is by sonication.

30: The method of claim 28 wherein the purifying is by filter chromatography.

31: A method of testing antigenic integrity of GP in a sample, comprising:
(i) detecting the presence or absence of a complex formed between anti-GP antibodies that bind conformational epitopes and GP in the sample, and anti-GP antibodies that bind linear epitopes and GP in the sample, and
(ii) comparing the amount of complexes formed such that the presence of an equal amount of complexes from both antibodies indicates antigenic integrity of GP, and a reduced amount of complexes formed with the antibodies which bind the conformational epitope indicates loss of antigenic integrity of GP.

32: A kit for testing antigenic integrity of GP in a sample, comprising:
(i) one or more antibody that binds a conformational epitope on GP;
(ii) one or more antibody that binds a liner epitope on GP; and
(iii) ancillary agents for detecting complexes formed between the antibodies and the GP in the sample.
